(19) United States
(12) Patent Application Publication
(10) Pub. No.: US 2025/0387044 A1
(43) Pub. Date: Dec. 25, 2025

(54) PERSONALIZED PHYSICS ENGINE

(71) Applicant: LogicMark, Inc., Louisville, KY (US)

(72) Inventors: Chia-Lin Simmons, Moraga, CA (US); Rafael Saavedra, Cupertino, CA (US); Peter Williams, Belmont, CA (US)

(21) Appl. No.: 19/247,428

(22) Filed: Jun. 24, 2025

Related U.S. Application Data
(60) Provisional application No. 63/663,390, filed on Jun. 24, 2024.

(51) Int. Cl. A61B 5/11; A61B 5/00; G16H 20/30; G16H 40/67

(52) U.S. Cl. CPC A61B 5/1113; A61B 5/002; G16H 20/30; G16H 40/67

(57) ABSTRACT

Personalized Physics Engines (PPE) are provided via personalizing a musculoskeletal representation for a person under monitoring (PUM) for care according to a Health Care Plan (HCP) to represent a movement capability of the PUM; receiving data from a sensor enabled environment (SEE) to identify behaviors of the PUM in the SEE; identifying an intent for a first series of performed behaviors of the PUM; modeling a series of predicted behaviors of the PUM via the PPE based on the intent and a current behavior of the PUM; identifying a second series of performed behaviors of the PUM; identifying a variation between the second series of performed behaviors and the series of predicted behaviors that satisfies an actionable threshold; in response to identifying that the variation satisfies the actionable threshold, engaging a hardware device associated with the SEE identified based on at least one of the intent and the variation.

### CROSS-REFERENCES TO RELATED APPLICATIONS

The present disclosure claims priority to U.S. Provisional Patent Application 63/663,390, filed on 2024 Jun. 24, titled “PERSONALIZED PHYSICS ENGINE”, which is incorporated herein in its entirety.

### BACKGROUND

Various sensors may monitor a sensor-enabled environment (SEE), and these sensors may provide data about the environment to one or more computing systems for analysis. A physics engine may be a computerized model of physical behavioral aspects of the world. Physics engines may model various forces upon an abject, both external and internal, and model a behavior of that object in response to various conditions and force applications.

### SUMMARY

Systems, methods, and apparatuses are provided for implementing a personalized physics engine. In an example, a system comprises one or more sensors configured to monitor and measure behaviors of a person under monitoring within a sensor-enabled environment, a memory, and a processing device configured to receive data from the one or more sensors, execute a physics engine configured to model one or more physical characteristics of the person under monitoring, wherein executing the physics engine includes determining a movement of the person under monitoring based at least partially upon the data from the one or more sensors, validate a detected movement by comparing an output of the physics engine with the data from the one or more sensors, and alert a third party that the movement has been detected.

### BRIEF DESCRIPTION OF THE FIGURES

Various example embodiments are provided in the following Figures and the detailed description thereof which follows below. It will be appreciated that these Figures are provided for illustrative purposes only, and that embodiments of the present disclosure are in no way intended to be limited by inclusions or exclusions of particular features within the various Figures herein.

FIG. 1 illustrates an example personalized physics engine, according to example embodiments of the present disclosure.

FIG. 2 illustrates an example of the joints of a limb, according to example embodiments of the present disclosure.

FIG. 3 illustrates an example of one or more movement modules, according to example embodiments of the present disclosure.

FIG. 4 illustrates an example where each of several joints and the respective orientations are represented by movement modules, according to example embodiments of the present disclosure.

FIG. 5 illustrates an example of movement of a set of joints which in various orientations are represented by mobility modules, according to example embodiments of the present disclosure.

FIG. 6 illustrates an example of a sensor-enabled environment where a person under monitoring is monitored by one or more sensors, devices, or systems present in the sensor-enabled environment, according to example embodiments of the present disclosure.

FIG. 7 illustrates a first example of a sensor-enabled environment with two areas, according to example embodiments of the present disclosure.

FIG. 8 illustrates a second example of a sensor-enabled environment with two areas, according to example embodiments of the present disclosure.

FIG. 9 illustrates an example of a person under monitoring who is located at a sofa which is in a sensor-enabled environment, according to example embodiments of the present disclosure.

FIG. 10 illustrates an example of a person under monitoring in a sensor-enabled environment with an initial start point undertaking a set of movements which are characterized by a set of movement frameworks, according to example embodiments of the present disclosure.

FIG. 11 is a flowchart of an example method for employing a personalized physics engine for improving the care of a person under monitoring in a sensor-enabled environment, according to embodiments of the present disclosure.

FIG. 12 is a flowchart of an example method for configuring one of both of the musculoskeletal models and sensor configurations used in monitoring and caring for a person under monitoring in a sensor-enabled environment with respect to a personalized physics engine for improving the care of the person under monitoring in a sensor-enabled environment, according to embodiments of the present disclosure.

FIG. 13 is a flowchart of an example method for maintaining and adjusting a healthcare plan for the monitoring and care of a person under monitoring in a sensor-enabled environment and adjusting a personalized physics engine accordingly for improving the care of a person under monitoring in a sensor-enabled environment, according to embodiments of the present disclosure.

FIG. 14 is a flowchart of an example method for supplementing gaps in observed behaviors of a person under monitoring in a sensor-enabled environment in data received from at least one sensor with data generated by a personalized physics engine for improving the care of a person under monitoring in a sensor-enabled environment, according to embodiments of the present disclosure.

FIG. 15 illustrates an example computing device, as may be used to provide one or more of the personalized physics engine, sensors, modules, or other systems described with respect to the sensor enabled environment or monitoring the person under monitoring, according to embodiments of the present disclosure.

### DETAILED DESCRIPTION

This disclosure describes the use of physics engines, which are based on the rules and laws of physics of the real-world environment that can be calibrated and configured to represent, at least in part, a person under monitoring (PUM) in a sensor enabled environment (SEE).

### PUM SIMULATIONS AND REPRESENTATIONS

Representing a PUM through the measuring of their movements, including sequences thereof, represented, for example as patterns, behaviors and other characteristics employing one or more physics engines configured to represent the real world physics of the movements of a PUM and their environment, offers several benefits and improvements in the systems used on monitor the PUM.

In some embodiments, a representation, including one or more models of a PUM, may be instantiated via a personalized physics engine (PPE) that represents that PUM in one or more SEE. For example, one or more sensors, devices or systems having been initialized, calibrated and configured may generate a set of data that represents a PUM, expressed for example, as the movements of the PUM, including patterns thereof, behaviors, or other activities in the form of a simulation. Such a simulation may include, for example, one or more physics engines that are configured to represent the physical characteristic or behaviors of a PUM. This representation can include the use of the measurements and other monitoring data of the SEE in any arrangement.

There are a number of available physics engines, such as for example, ODE (Open Dynamic Engine), that can be used to represent the physics of human movement. There is considerable complexity involved when a high degree of accuracy of those movements is desirable, and there are several simplifications that can be employed, including the reduction of a 3D spatial model to a 2D planar model, the reduction of image data to line drawings, reduction of precision, reduction of multidimensional data sets to less dimensions, and the like.

In some embodiments, muscular skeletal models, embodied via physics engines, can be employed where the movements of a PUM can be represented as can the forces employed by that PUM in making such movements.

In the context of representing a PUM in a SEE, the physics engine may be configured, at least in part, through the use of calibrations, to include pattern frameworks, patterns or behaviors, as the set of movements a PUM can undertake is constrained, at least in part, by the age, physical condition, and any other constraints on the abilities of the PUM. For example arthritis and the like may constrain the movement of a PUM. For example, a PUM confined to a wheelchair, missing a limb, having a joint (e.g., in the spine) fused, in a brace or cast placed on a joint or limb, or the like may be physically constrained in the movements possible compared to other persons. For example, configurations may also identify behaviorally constrained movement sets, such as a PUM in their sixties or seventies is highly unlikely to doing the splits, and if that PUM does do a split, the actions is highly likely (relative to a younger PUM) to be a detrimental health and wellness situation for the PUM.

One aspect of this approach may be the use of multiple sensors, devices or systems, where a set of these is monitoring the PUM in the SEE, providing a range of data sets that represent the patterns of behaviors of the PUM. This approach reduces the complexity of the modelling of the representation of the PUM, as many of the activities of the PUM follow patterns identified, at least in part, by the data sets generated by the one or more sensors, devices or systems deployed in the SEE.

In some embodiments, one or more topologies representing real-world conditions may be employed to represent the PUM. For example, a human muscular skeletal representation, where each of the joints is represented as range of possible movements in three physical dimensions and time, the distance between the joints is represented by a further range and the like. This representation can include physical constraints, such as the rotation of the neck at a maximum of 180 degrees of movement. In some examples, this representation may be further personalized for the PUM, so that a baseline representation of neck rotation of 180 degrees is constrained to be less than 180 degrees for a PUM in a neck brace or expanded more than 180 degrees for a PUM noted in a health care plan has having hyper-flexibility in the neck joint. There can be other sets of constraints that are configured, which may, at least in part, be determined from a health care profile of the PUM. This configuration can include the sensors, devices or systems of the SEE measuring the movements of the PUM as a basis for configuration of a physics engine, and, in some embodiments, can include the use of specific measuring systems to establish the movement capabilities of a PUM.

The use of topologies may include, for example, one or more sets of configurations such as torque, ranges of movements (for example a knee can only bend in certain directions without damage), height, weight, age or other attributes of a PUM or other physical metrics, which can be represented as multiple dimensions in and of one or more topologies.

In some embodiments a set of agility metrics may be employed, through for example calculation based, at least on part, by observation and measurements of the one or more sensors, devices or systems present in a SEE or by declaration, based at least in part on multiple observations and measurements of other PUM in other SEE with similar attributes. These metrics can represent the degree of agility a PUM may have in undertaking one or more movements, including sequences thereof.

The need and degree of accuracy of these representations may, at least in part, be determined by an initialization, calibration and configuration of the one or more sensors, devices or systems deployed or present in the SEE or the one or more care hubs or care processing systems. These parameters can include, for example, the fidelity and granularity of the measurements represented by the data sets generated by such. Unlike when physics engines and musculoskeletal systems are employed for surgery, robotics or other employment scenarios where high accuracy is essential, the systems may be deployed herein for the purpose of monitoring the health, wellness, care or safety of a PUM, with an accuracy that is sufficient to determine the movements being undertaken and the relationship of these movements to the behaviors of the PUM in those circumstances.

In that regard, these representations can employ simplifications that enable the one or more care hub or care processing systems to identify those situations that are detrimental to the health, wellness, care or safety of the PUM. These determinations can include the use of one or more metrics, for example, risk metrics that represent the actual or potential risk of one or more movements, patterns, behaviors or other actions or events to the wellness, health, care or safety of the PUM in a SEE.

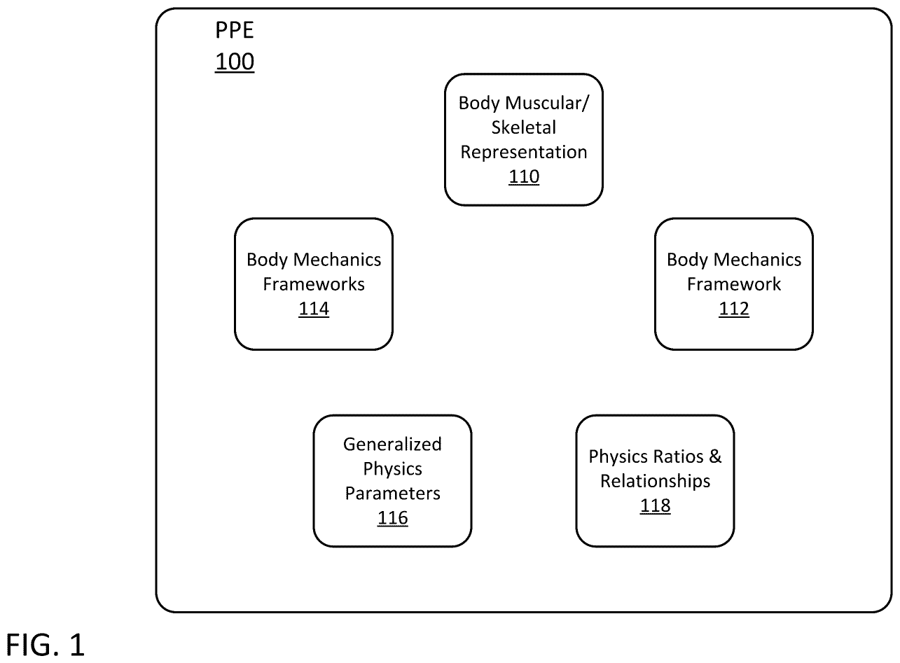

FIG. 1 illustrates an example embodiment of a personal physics engine (PPE) 100, which includes a body muscular/skeletal representation 110, a body mechanics framework 112, body movements framework 114, generalized physics parameters 116, and physics ratios and relationships 118; each of which may be configured to represent a PUM.

For example, a sequence of movements may involve sets of joints operating in series or in parallel, for example the movement from sitting to standing may involve ankles, knees and hips as the legs move and hands, elbows, and shoulders as the person lifts themselves from the sitting position. Similarly, when walking, there can be a sequence of movements involving each joint in a particular sequence.

In some embodiments, a behavior, such as walking to the kitchen, includes a sequence of movements, represented for example by mobility frameworks that can be populated by the one or more data sets generated by one or more sensors, devices or systems in a SEE. This sequence can be represented as a set of such quantized movements, and one or more care hub or care processing system may be employed to ascertain any deviation from this sequence. For example, if a PUM starts on a movement sequence where the likely target is the kitchen, and then retraces their steps and picks up an object (e.g., a pair of glasses), this may, for example, be evaluated as a continuation of the sequence. In contrast, if the PUM undertakes this retracing of steps multiple times, this repeated behavior may represent confusion of the PUM, and lead to a change of state being identified or one or more alerts being generated. For example, a PUM suffers a dementia event, identification of the repeated back and forth pattern of movement may be used to identify the dementia event via the physical movements of the PUM being indicative of a mental event.

However, sensors in the SEE may be obscured, have blind spots, go offline, lack sufficient granularity to detect an event, or be focused away from an event, which may prevent direct detection of the movements of a PUM. A PPE can be used to supplement or fill in any gaps of coverage from the sensors in such cases. For example, if a sensing apparatus is obscured, a PPE may use a model of the PUM to calculate the data set of one or more sensors, devices or systems where an interpolation mechanism, for example a camera or other directional sensor, does not have an uninterrupted point of view of the PUM and their movements. The PPE may estimate the data sets such sensors, devices or systems may generate and integrate this estimate with the actual generated data set.

The alignment of the data sets generated by the SEE to a framework that includes a physics engine that is, at least in part, configured to represent the movement and dynamics of the human body, can be undertaken on, for example, calibration, snapshot, accumulation or other basis, to create a personalized physics engine representing a specific PUM.

In some embodiments, a physics engine that, for example, includes a general model of the muscle and skeletal framework of a human body can form, at least in part, the basis of an AI/ML model, for example employed by an LLM/LCM. This general model can include where the physics engine, although generic in nature, employs a set of body dynamics that are valid for all humans in 1G gravity, can be used as part of a model that represents a class of humans, for example those with specific Healthcare Plan (HCP), including for example, age, health conditions, recent procedures and the like or for a specific human. For example, the physics engines can provide several models that are usable as a baseline for different persons based on various characteristics that may be further configured and personalized for a specific PUM. For example, the physics engine may include a fist model for male PUMs and a second model for female PUM based on average proportions, joint spacing, and flexibility characteristics for an average male or female, which can differ between the two models. Further customization and personalization to the baseline models may allow for adjustments for height, weight, know movement patterns or constraints, and the like.

### ADJUSTING MODELS TO SPECIFIC PUMS

In some embodiments, the fidelity of recognition of the movement of the PUM can be aligned to the movements of the limbs and other moveable elements of a PUM's body (such as the head or neck), such that the relationships between the joints of these elements, for example, limbs, can provide sufficient data to represent the actual or potential movements of a PUM. In some examples, the data includes data as vectors than indicate the velocity or acceleration of the limbs.

One aspect of the PPE may be calibration of the underlying physics represented in the physics engine and the expression of those physics characteristics as employed by a PUM. For example, the determination of the degree of rotation of any one limb or joint of a PUM can be undertaken through one or more sensors, devices or systems being configured to identify and measure that rotation. This calibration can be achieved, to the degree necessary to evaluate the movement of joint or limb in the context of the behaviors of the PUM through, for example, a specific direct measurement, for example, employing sensors that are directly connected to that joint or limb, so as to establish the range or rate of movement, through either continuous or a set of snapshots of the joint or limb in various orientations. In some embodiments, for example, these measurements are evaluated, by the use of one or more skeletal and muscular models or one or more specialized techniques based on the skeletal and muscular representation of the human body that can, for example, be correlated to the age, condition or other physical aspects of the PUM. This calibration can include measurements on an accumulation basis, where the day to day movements of the PUM are evaluated using the data sets and patterns generated by the one or more sensors, devices or systems present or embedded in a SEE, in any arrangement.

A process of calibration may be undertaken on the initialization of a PPE for a specific PUM or may be repeated on a periodic basis. The calibration processing may align the underlying physics models and any human skeletal or muscular models with the conditions of the PUM such that the PPE can be deployed in one or more digital twins or other representations of the PUM to support, at least in part, the monitoring of the health, wellbeing, care or safety of the PUM.

This calibration may be used in support of the calibration or configuration of the one or more sensors, devices and or systems deployed or present in the SEE. For example, the calibration may be used also provide a bubble of sensing awareness, where the one or more sensors, devices or systems may be configured to focus on the movements of one or more joints or limbs, for example to refine the PPE representation of a PUM. For example, the reach of the PUM, that is the degree of movement they can accomplish, can form the boundary of such sensing bubble.

This sensing awareness bubble may also be used to determine the edge of the reach of the PUM as the PUM traverses through a location or undertakes a task, such as for example, getting a glass of water, or food from a refrigerator, such that any obstructions or other potentially adverse interactions may be mitigated or avoided, including through alerting the PUM (or a caretaker or other stakeholder in the care of the PUM), for example through visual, audio, haptic or other mechanisms.

This sensing bubble may also be used to evaluate the interactions of the PUM with the environment, for example the boundaries such as the floor, walls, windows and the fixtures and fittings such as furniture, appliances and the like. The physics engine may be used to, at least in part, determine the force of any interactions. For example, if the PUM puts a hand on a counter top, the force, velocity, or acceleration of the movement can indicate the intention of such movement. For example, if the PUM is simply resting a hand on the counter, then the force, velocity or acceleration is minimal or within existing thresholds of the patterns of movements for that PUM. However if the PUM used the counter to steady themselves, such as to prevent or catch a fall, then the force, velocity or acceleration will be higher, potentially exceeding one or more thresholds. In the latter case, the systems monitoring the PUM may raise an alert or communication, including to the PUM or a caretaker or other stakeholder in the care of the PUM, for example suggesting the PUM sit down or enquiring as to the condition of the PUM. A further example is where a PUM has taken a medication (and such event is recognized by the one or more sensors, devices or systems of the SEE, and may form part of the HCP), and subsequently has become light headed and used the counter or other solid surface to steady themselves.

Within such a sensing bubble, the orientation of the PUM, in terms of the limbs and movements of the PUM, can include the trajectory of those movements. For example, when a PUM is walking, moving each leg in a consistent trajectory, the one or more sensors, devices or systems in a SEE can measure such movements and identify that these movements form part of a pattern; that is walking in a manner that is part of a quiescent state. However, if one leg is on a trajectory that is not part of this pattern, the monitoring systems may generate a communication or alert to the PUM or other stakeholders. This deviation from the pattern of movement from the quiescent state may indicate a fall event, a newly developed limp or other injury affecting gate, a sign of a stroke, or other healthcare condition for the PUM.

In some embodiments, a simulation can initially be generated in the form of a line representation of the PUM, for example based on the body of the PUM represented in the form of a simplified model such as a stick figure, which can then be populated by the various data sets generated by the one or more sensors, devices or systems and organized as the pattern frameworks and behavior patterns as the behaviors and patterns unfold. These patterns, expressed in some embodiments as tokens, can form a model of the PUM that can be used in one or more digital twins, in conjunction or collaboration with one or more artificial intelligence (AI)/machine learning (ML) modules, including LLM (Large Language Model)/LCM (Large Concept Model), to monitor or predict the PUM patterns or behaviors.

In some embodiments, a personalized physics engine (PPE) may be employed, which can be initialized and configured to represent the PUM through the measurement of the physical characteristics of the PUM. These measurements may be specified using, for example, particular activities undertaken by the PUM, for example walking, sitting, raising an arm or the like, or may be measured using the sensors, devices or systems, including worn, carried or implanted, of the SEE as the PUM carries out day-to-day activities.

One aspect of a PPE may be training of the PPE with the movements of a specific PUM. The PPE can represent the physical attributes and characteristics of a PUM, such as for example, gait, arm or leg movements, back, neck and other joint movements, stoop and any other measurable physical characteristics, including gaze tracking. These characteristics may be measured through the capabilities of the SEE or any sensors, devices or systems employed for that purpose, including with specialized sensing systems, such as full height mirror surfaces with embedded sensors, such as cameras, radar, light detection and ranging (LIDAR) or other active or passive sensing capabilities. In this manner, a PUM may interact with such a sensing system facing, for example front, side and back to the sensing system to create a 3D image of the physical body of the PUM.

In some embodiments, these measurements may be undertaken by the one or more sensors, devices or systems present in the SEE, where one or more of these sensors, devices or systems create a data set that represents part of the of PUM movement characteristics and these sets are combined with a generic physical model of a human, that includes skeletal and muscular representations to create an individual physics based movement representation of a specific PUM.

In some embodiments, these approaches may be used in combination. For example, a 3D model of the PUM may be created using a sensing system, to create a PPE of that PUM, which may then have additional data sets, generated by the one or more sensors, devices or systems present in a SEE, that modify the PPE in response to consistent changes in the physical attributes of the PUM. For example, if the PUM has undergone a procedure, for example a hip replacement, the PPE can be configured to accommodate such change, for example by variations in the HCP or by measuring the changes in the mobility of the PUM. These changes may then be compared, for example, with the anticipated changes such a procedure produces, for example the range and constraints of mobility as the healing process occurs. In this manner, the progress of a PUM may be compared to the specified progress and any variations identified. These variations may then inform alerts or communications to one or more stakeholders, including the PUM, to assist in the recovery of the PUM.

In some embodiments, patterns, for example those based, at least in part, on data sets generated by the one or more sensors, devices or systems present in a SEE, may individually or in any arrangement form behavior tokens, named bevokens, that can represent, for example Time of Day (ToD) or quiescent state or event behaviors represented as tokens.

This aspect of the system can represent events, actions or other activities as sets in the form of patterns, where in some embodiments these patterns may, in whole or in part, form one or more behavior tokens, described herein as bevokens, representing the behaviors of, for example, a PUM. Accordingly, the token or bevoken can identify, via a first level of encryption, an identified behavior, while including at a second level the data used to generate the determination. Accordingly, various systems may use the first determination (e.g., the behavior) without having to access the underlying data or earlier determinations, thereby improving data privacy while maintaining a high level of care and reducing the use of computing resources, while still offering those systems that require the underlying data (and have permission to access the data) access to the data, and a way to more efficiently identify the relevancy of the underlying data. For example, a first system with a first permission level may access a first and a second token to identify two identified behaviors of a PUM, but not be permitted access to the video or other data of the PUM used to identify those behaviors. Similarly, a second system with a second permission level sufficient to access the underlying data may access the first and second tokens to identify two identified behaviors of the PUM and then determine which sets of underlying data to access for further analysis based on the identified behaviors. For example, the second system may identify that a first behavior indicated by the first token of “sitting” is not of interest for further analysis, and foregoes accessing a first data set used to establishing the sitting behavior, but may identify that a second behavior indicated by the second token of “falling” is of interest for further analysis, and accesses a second data set used to establishing the falling behavior.

The PPE can also have a representation of the behaviors of the PUM as the behaviors can be represented by, for example, one or more tokenized behaviors, such as those aligned to one or more pattern frameworks, patterns or tokens or bevokens of that PUM. These representations of repeated behaviors of the PUM, can at least in part, form part of the PPE, where, for example the mental state of the PUM, as represented, at least in part, by these tokens, and can then be evaluated or determined, for example, using one or more care hubs or care processing systems.

The coupling of the PPE with one or more AI systems, including systems using LLM/LCMs, supported by one or more digital twins of a PUM, where such digital twin can represent physical actions or behaviors that a PUM may undertake in a SEE. This representation can include predicting behaviors, actions or events that are possible in a digital twin of the SEE with a digital twin of the PPE of the PUM, so as to create predictive representations of such activities as the activities may impact the care and wellness of a PUM. For example, such digital twins can include representations of a sensing bubble, which can be used to anticipate the potential interactions of the PUM with the environment of the SEE. In this manner, potential interactions where the health, wellness, care or safety of the PUM may be impacted can be identified. For example, this identification may result in removing an object, for example a chair or other furniture, from the path a PUM may take, for example when going to the bathroom at night.

In some embodiments, one or more recurrent neural networks may be invoked so as to use feedforward and feedback networks. For example, with a feedforward network, the patterns generated by the one or more sensors, devices or systems may be represented as functions connected as a chain. In this manner the neural network may act to match the behaviors, represented as the outcome based on patterns representing training data.

For example, if the digital twin of the PPE generates a movement pattern of a PUM, for example, a decrease in the use of the left arm, for example through data representing the observation that the PUM rarely picks up an object with the left arm, even though the object is on the left of the body of the PUM, this simulation could indicate a health, wellness or care event, such as lack of mobility on the left side, which could indicate heart troubles, eyesight difficulties and the like. The recognition of such behaviors can be communicated to one or more care processing systems, including care hubs, one or more stakeholders or the PUM.

In some embodiments, a shower facility with embedded sensors may be used to capture body features of a PUM. For example, a shower facility may include one or more active scanning devices, for example laser, LIDAR, radar or the like. When the shower is initialized and calibrated, the water flow from the shower head may be set to a specific, temperature, size or rate and the water flow, including the dispersion pattern caused by the body of the PUM, may be measured by the one or more active scanners. For example, when the PUM is using the shower, the interference pattern created by the water dispersed by the body of the PUM in the spray path from the showerhead may be scanned using the one or more active scanners so as to determine, at least in part, an accurate representation of the body of the PUM. These data may then be communicated to a PPE and be employed, at least in part, in the PPE for representing the PUM.

In some embodiments, a PPE can form part of a monitoring system, which includes one or more AI/ML systems, including LLM/LCM. These AI/ML systems may use training data generated by the one or more sensors, devices or systems present in a SEE to predict the potential movements of a PUM. In some embodiments, a PPE may be used as part of a retrieval-augmented generation (RAG) model, where the PPE provides data sets that represent the real-world physics of the environment, which can include, at least in part, a musculoskeletal representation of the PUM, including the relationships between the joints and other flexible body elements, as context to the AI/ML systems, so as to ensure the predictions of the AI/ML systems are aligned the capabilities and characteristics of the PUM.

In some embodiments, a generative adversarial network (GAN) may be employed with a PPE to evaluate, validate or determine potential outcomes, from the one or more AI/ML systems, including LLM/LCM, or the one or mor sensors, devices or systems.

In some embodiments, training data sets can include large image models derived, at least in part from multiple image sources, potentially classified by similarity to one or more PUM. For example, an agent based approach may be employed using, for example, directed acyclic graphs that are based, at least in part, on images and sequences thereof of one or more PUM in a SEE. These images may be reductionist versions of the captured data, where, for example, a PPE provides a musculoskeletal framework to which the image, using one or more AI systems, including LLM/LCM, is compared to find a best fit reduction of the joints of the PUM in that image. This approach can provide the data for movement modules or mobility frameworks without compromising the privacy of the PUM.

In some embodiments, such an approach can be deployed across multiple PUM to create a sets of movement modules or mobility frameworks that can be aligned to the specific conditions of the one or more PUM. This alignment can include those conditions that are aligned with one or more procedures that a PUM may experience, for example hip replacement, knee replacement and the like.

In some embodiments, there may be a calibration phase for a PPE, where for example, one or more tests are undertaken to establish one or more baselines for a PUM that the PPE can represent. For example, these tests can include but are not limited to: eyesight tests using, for example smart TV or other display device(s); simulated reaction testing, where the image of an obstacle is projected towards a PUM; temperature testing, including using heating ventilation and air conditioning (HVAC) systems or other heat or cold sources; active capture processing, including millimeter (mm) radar, LIDAR, Video and the like; integration with one or more appliances or devices or the like; neural networks; and the like.

### HEALTH OR PERSONAL SAFETY

In some embodiments, a PPE can include, by reference or embedding, a set of sensors, devices or systems including one or more carried, worn or implanted sensors, devices or systems. The PPE can include the use of one or more repositories which can be used by the PPE in whole or in part. The PPE can incorporate dynamic additions of authorized and authenticated sensors, devices or systems. In some embodiments, tokens can be used for the communication of identity, authentication or access information or one or more data sets generated by one or more sensors, devices or systems.

For example, a PPE may be functionally distributed across multiple sensors, devices or systems, where if a PUM is in a sitting position, a worn device may be employed, at least in part, to identify hand or arm motions. For example, data sets from different sensors, devices or systems may be given differing priority, weighting or precedence based, at least in part on the current context of the PUM, for example sitting. This prioritization enables the identification of quiescent motions, such as turning the pages in a book, swiping on an electronic device, touching the face or other movements that are of no or little consequence in that context of a quiescent state. This prioritization can include those motions that may interact with other environment elements, such as furniture, lamps and the like, where these interactions, such as sitting on a couch, turning on a light, and the like are also part of the quiescent state.

In one example scenario, a PPE may integrate data from various sensors, devices or systems present in a SEE, for example, the sensors may include a worn accelerometer affixed to a limb of the PUM (such as might be found on a smart watch) and one or more mm radar or LIDAR scanners monitoring a physical space of the SEE, for example smart lightbulbs or specialized sensors. Data from these sensors may be fed to a recurrent neural network (RNN) trained on historical data from one or more PUMs, which may include historical data from this specific PUM. Data from the one or more sensors, devices or systems may also be used to continuously train the RNN, which may take the form of in-place training, where the RNN is incrementally or continuously trained with incoming data, or full-dataset retraining, where the recurrent neural network is periodically completely or partially retrained with an updated data set.

The RNN may process sensor, device or system data sets as inputs to modify one or more digital twins representative of the PUM to predict one or more possible motions of the PUM from a current position or context of the PUM. The one or more digital twins may include one or more simplified representations of the PUM modifiable by the RNN such that one or more motions may be modeled in parallel to produce three-dimensional representations of the PUM during or after the one or more motions. The digital twins may also include data about mass distribution on the one or more simplified representations and calculations of forces exerted upon one or more parts of the one or more simplified representations. For example, a PUM wearing a cast on one arm may be modeled in a digital twin as having more mass on that side of the body than in a digital twin modeling that PUM without a cast.

In this example scenario, a PUM may begin to fall down. The one or more sensors, devices or systems present in the SEE detect the change in orientation of the PUM and may send tokens that represent this behavior (based on the detected data) to the PPE. The PPE may detect a change in motion of the PUM and model one or more trajectories for the impending fall. The PPE may detect that, based upon data calculated from manipulating the digital twins, the PUM is likely to suffer an arm breakage from forces exerted by a landing after the fall. The PPE may thus, for example, alert emergency services to the fall before the PUM contacts the ground, thereby affecting a rapid response time.

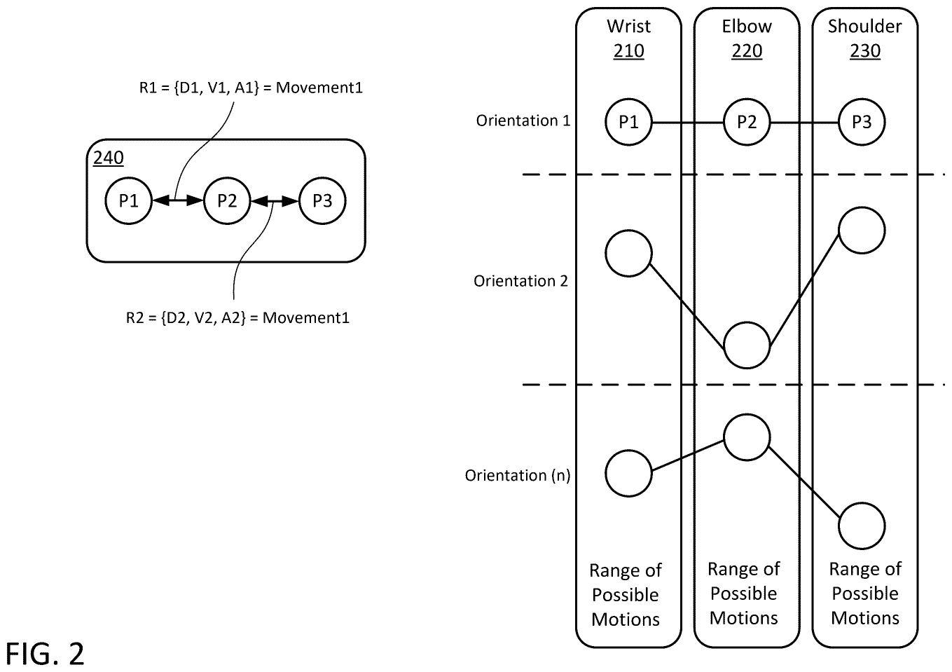

FIG. 2 illustrates an example of the joints of a limb, in this case an arm with wrist 210, elbow 220, and shoulder 230. Each of these joints may be in various orientations, for example orientation 1, 2 or (n). However, as the PPE includes a musculoskeletal model of the human body, these orientations comply with that model and are likely to have range of motions that are subsets of the possible range of motions described by the model. In some embodiments, there can be relationships between the joints, illustrated in movement 240 as relationship R1 (between the wrist 210 and the elbow 220) and relationship R2 (between the elbow 220 and the shoulder 230). Each of these relationships can include at least one vector indicating the direction and alignment of the joint to the other joints and potentially to the 3D environment in which the PUM is domiciled. These vectors may include, for example, directional (e.g., D1, D2, etc.), velocity (e.g., V1, V2, etc.) or acceleration (e.g., A1, A2, etc.) data.

In some embodiments, the interactions of joints and muscles can be considered in the form of a game theory game, where only certain outcomes are valid or possible. For example, the rotation of the head cannot traverse 360 degrees. This approach can be further deployed using the pattern frameworks, patterns or behaviors of a PUM, where the range of activities a PUM may undertake in a SEE are relatively constrained and can be expressed as games employing game theory. This framework is particularly the useful where, for example the one or more sensors, devices or systems deployed or present in the SEE are configured to use one or more game theory games to, at least in part, identify the one or more patterns or behaviors of the PUM that are represented by the data sets of the one or more sensors, devices or systems.

Although the examples presented herein provide for the positions and relationships of joints in the arm, the present disclosure contemplates that various other joints and body parts may be monitored by the PPE via the relationships and movement vectors thereof. These joints may include various large joints (e.g., hip, knee, ankle, shoulder, elbow, wrists, etc.) and small joints (e.g., fingers, toes, jaw), and describe relationships for adjoining joint parts (i.e., a first joint relative to a second joint with no intervening third joint; e.g., a relationship of a left wrist to a left elbow joined by a left forearm) or non-adjoining body parts (e.g., relationship of a left wrist to a right wrist). Various joints may be considered individually, collectively or with various subdivisions. For example, a spine, may be represented by various individual joints between each vertebra, collectively as a single joint, or as subdivisions of a neck joint and a lumbar joint. For example, a hand may be represented by various individual joints between the bones thereof, or may be represented as a collective joint having various states (e.g., open, resting, grasping), as an extension of the wrist joint (e.g., ignoring the small joints), or with certain fingers or the palm treated as groups (e.g., a first model of the palm, thumb, index finger, and non-index fingers, a second model of the palm, thumb, and non-thumb fingers). Ranges of motion considered for each joint may include abduction, adduction, circumduction, rotation, pronation, supination, flexion, extension, inversion, eversion, occlusion, protrusion, and combinations thereof. The PPE may identify acceptable or expected ranges of the various motions based on a general framework for a person at each joint, which may be modified according to the HCP for the PUM (e.g., to identify reduced ranges of motion due to injury, amputation, joint fusion, arthritis, individual flexibility, etc. or extended ranges of motion due to injury, amputation, individual flexibility, etc.). Accordingly, the PPE is may be configured in various embodiments to use different collections of joints 250a-250n (generally or collectively, joints 250) according to the PUM, the SEE, and level of detail offered by the sensors at any given time.

For example, the PPE may use a first model of the PUM while the PUM is in a first state (e.g., sitting on a sofa) that uses a first number of joints 250, but switches to a second model of the PUM what uses a second number of joints 250 when the PUM switches to a second state. In some embodiments, number of joints 250 used in a new model increases relative to a previous model based on a detected task/intention of the PUM (e.g., adding additional joints for a hand and fingers thereof when a grasping task is identified), sensor abilities (e.g., individual leg joints may added when a PUM removes a blanket from the legs, thereby rendering individual leg tracking possible) or potential injury to the PUM (e.g., adding a collar joint between an existing neck joint and shoulder joint in response to a potential fall to diagnose whether a clavicle has been broken). In some embodiments, the number of joints 250 used in a new model decreases relative to a previous model based on a detected task/intention of the PUM (e.g., foot-ankle joints may be removed when a PUM changes from a walking state to a sitting state and removes weight from the feet), sensor abilities (e.g., individual leg joints may be combined when a PUM places a blanket over both legs, thereby rendering individual leg tracking impractical), or injury (e.g., an elbow 220 may be removed between a wrist 210 and shoulder 230 (or have a range of motion set to zero) when the PUM is wearing a cast that immobilizes the elbow 220).

### MOBILITY FRAMEWORKS

Many of the patterns, behaviors and characteristics of a PUM involve, at least in part, movement. These movements are predictable to a large degree within the context of a PUM domiciled in an environment, for example a SEE. As a PUM is a bipedal human, the range and scope of movements is finite and to a large degree can be represented, at least in part, by one or more patterns or behaviors. These behaviors can be represented through data sets or patterns generated by the one or more sensors, devices or systems that are present in a SEE, which can include a PPE, which can be configured to represent, at least in part, the movements of the PUM.

In some embodiments, the PPE can include a set of frameworks that can represent the typical motions of a PUM. For example, these frameworks can include motion-based activities such as walking and running, stationary activities such as sitting and lying, and partial motion activities such as picking up objects, reaching for objects or specialized activities such as cleaning, cooking, waste removal or other recognizable behaviors. Each of these activities may be represented by a framework which includes, at least in part, the relative motion of the joints of the body and the relationship of the joints to each other. For example, walking involves each leg swinging from the hip, up to a certain angle, resulting in each foot being placed in front of each other. Similarly, a sitting position involves at least one angle between the torso and the legs.

In some embodiments, each of these frameworks can be expressed as a set of relationships between the joints or torso of the body, where those relationships are representative of a typical PUM, for example based on one or more PUM's age, physical characteristics and the like. These frameworks provide the PPE or one or more AI/ML systems with a set of data that can be used, at least in part, to ensure that representation of the PUM is suitably correlated to the actual PUM behaviors as detected by the one or more sensors, devices or systems involved in monitoring the PUM. This correlation can be configured, at least in part, by calibration of the PPE as described herein.

These frameworks may include a set of framework elements, such as stretching an arm, where these elements represent simplified modules of movement of one or more PUM, described as movement modules. For example, using a hand to grab an object where the thumb and fingers grip the object, may be such a module. These modules may be combined to create more complex movements, such as walking, sitting, exercising or the like. These modules can be used to identify data sets from the one or more sensors, devices or systems that are not physically possible, such as a hand attached to a leg. This approach is particularly useful when one or more parts of the PUM, for example a leg, is obscured, from the perspective of one or more sensors, devices or systems, by an arm, such as when reaching for an object on the floor, where the sensor, device or system data sets is not an accurate representation of the physical movements of the PUM.

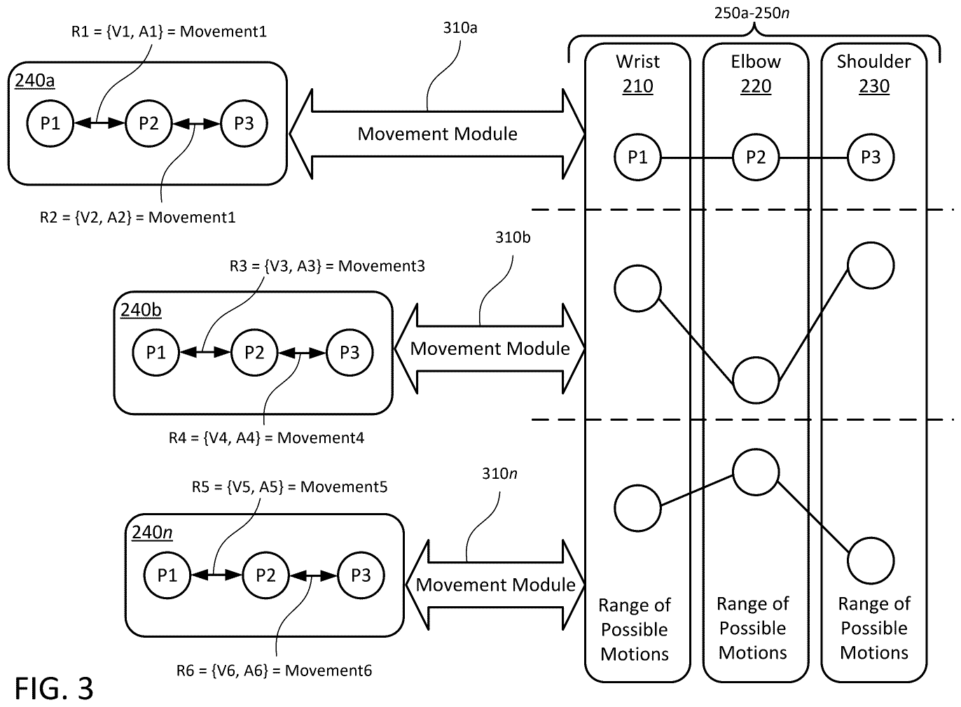

FIG. 3 illustrates an example of one or more movement modules, where each of the joints, for example, wrist 210, elbow 220, and shoulder 230 have specific relationships represented by movement modules 310a-n (generally or collectively, movement modules 310). These movement modules 310 may include the relationships (e.g., R1, R2, R3, etc.) between the joints including vectors or movements 240a-n (generally or collectively, movements 240). In various embodiments, for an individual PUM with known distances between certain joints (e.g., the length of a forearm between a wrist 210 and elbow 220 on the same arm), the distance between the joints may be omitted from the vector relationship (or be included and ignored) in a movement 240, while other joints may include a distance measurement as part of the movement 240 when the distance may be variable (e.g., a distance between a left wrist and a right wrist).

The use of modules enables the PPE and AI/ML systems in combination to use predictive models that can identify, at least in part, the movements of the PUM, which in turn may correlate with the intentions or attentions of the PUM as described herein. This approach can include the use, for example, of recurrent or context aware neural networks where feedforward and feedback techniques are employed. In some embodiments this approach can include the use of one or more game theory games, for example, to validate the AI/ML systems outputs or such systems' predictions of the one or more data sets or patterns generated by the one or more sensors, devices or systems.

In some embodiments, these frameworks may be stored in one or more repositories, where for example the one or more frameworks may be configured to represent differing mobility and other physical characteristics, based at least in part on one or more PUM characteristics. For example, these frameworks may be organized by age, mental acuity metrics, medical conditions as specified in an HCP, medications being taken (including the side effects or other classification schema of those medications), including those determined, in whole or in part by one or more AI/ML systems, including LLM/LCM.

In some embodiments, a PPE may employ one or more models of one or more PUM that include joint alignment or measurements thereof. For example, a PUM may be measured by one or more active or passive sensors to identify the positions of the joints of the PUM, for example, elbows, wrists, knees, ankles, neck and the like to confirm the relative distance between these joints or the potential vectors of the movements of these joints. These measurements can then be used to, at least in part, determine the radius of reach and length of step of the PUM, which can be represented by a movement bubble or PUM space encompassing the potential movements of any of the PUM limbs in any direction that they are capable of.

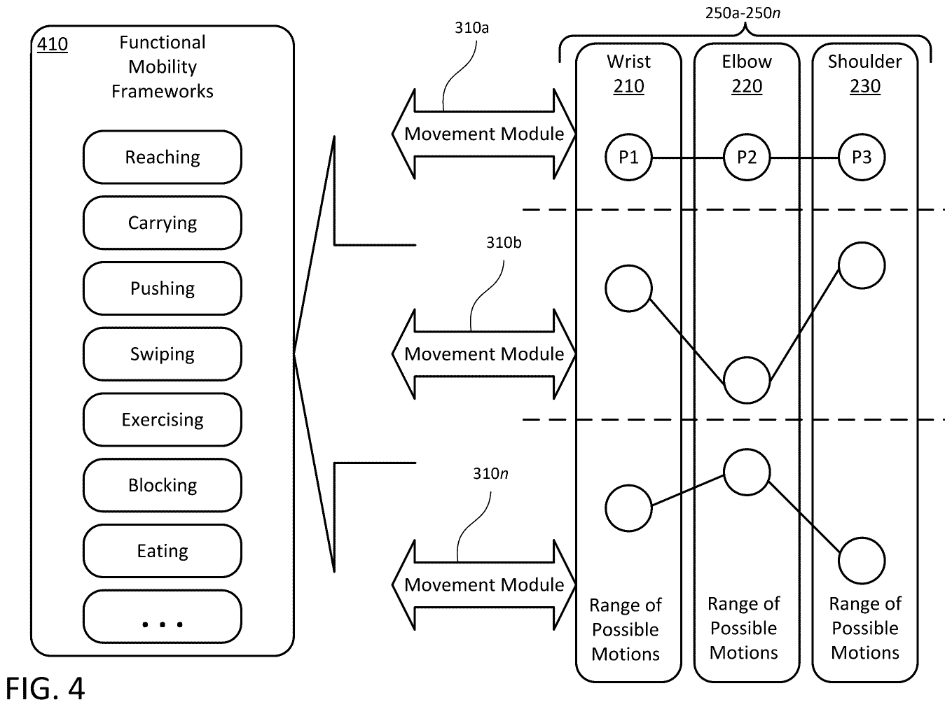

FIG. 4 illustrates an example where each of the joints 250 (e.g., wrist 210, elbow 220, and shoulder 230 and the respective orientations are represented by movement modules 310 and as such can form, at least in part one or more functional mobility frameworks 410.

For example, a PPE may use these measurements to determine, at least in part, the space that the limbs of a PUM can occupy. This determination can include determination of possible intersections of that space with fixed or mobile artifacts that are present in the SEE, for example, furnishings, kitchen and appliances and the like. This PUM space can be dynamic, in that as the PUM moves within one or more SEE, or potentially in other locations that have sensing capabilities, including those provided by carried, worn or implanted sensors, devices or systems, enabling the potential interactions of the PUM and the environment to be predicted, measured or calculated.

For example, if a PUM is walking in a garden or outside area, one or more sensors may be forward or downward facing to identify discontinuities in the path being taken. These discontinuities may be alerted to the PUM, for example though haptic or other methods, to avoid, for example, the PUM tripping over such discontinuities.

In some embodiments, the dynamic nature of a PPE can involve sufficient processing power so as to be able the monitor the movements of a PUM in space and time, and as such can involve one or more processing systems in arrangements that provide such sufficient capability, for example using a set of processors, such as those at the edge of processing environments, for example in sensors, devices or systems or those in a centralized or specialized processing environments, such as a care hub or care processing system or one or more cloud based processing systems or services.

In some embodiments, the data sets generated by these one or more sensors, devices or systems may be calibrated to the specifics of the PUM, for example as a token representing a particular action of a PUM. For example, tokens may represent behaviors such as taking a step, reaching for an object and the like, where these tokenized representations are conformant with the natural observed movements of the PUM. In some embodiments, these tokenized representations may be identified as quiescent (cf., actionable). In the same manner, any data sets representing an action of the PUM that is outside the thresholds for such quiescent states, may be identified as non-conformant, and can be used to identify potentially detrimental care, wellness, health or safety situations for a PUM. In some embodiments, framework or the modules thereof, may be represented by tokens, in any arrangement.

One aspect of the PPE can be the capability to quantize the relationship of the PPE to the PUM. For example, the PPE may represent a “snapshot” of the state of the PUM at a particular time and this snapshot may then be stored in one or more repositories. The timing of these snapshots may be determined, at least in part, by one or more care hubs or care processing systems. For example, if the overall health, care, safety and wellness of the PUM is consistent over an extended period or one or more predictions of that wellness, care, safety and health of the PUM indicate a consistent state, for example represented by a quiescent state, then the snapshots may, for example, be undertaken on a periodic, for example monthly basis. This timing basis may then be modified in line with any changes in the health, care, safety and wellness state of the PUM, for example by increasing the frequency, for example, to weekly.

In some embodiments, a PPE can have a set of frameworks representing the human body as a set of articulated entities, where each of these entities can be specified as representing a human of certain body types. These types may include one or more classification schema, such as gender, height, age range, mass, body ratios and the like. For example, body ratios may include various geometric relationships, such as hip to knee, knee to foot and the like, such that the length and range of motion can be determined.

This approach can include using multiple sensors, for example including, visual, radar, LIDAR, audio, haptic and the like such that the framework is populated with the data sets generated by these one or more sensors, devices or systems. These data sets can, for example, include data sets that represent the interaction of the framework representing a PUM and the environment, for example when a PUM sits on a chair, puts in or retrieves food from a refrigerator or the like.

There are many physics engines that are designed to model the interactions between objects, generally collisions, and these physics engines are used in many games, however the use of such techniques can be applied to the interactions of the representation of the PUM, which includes the PPE for the PUM, and the environment of the PUM or the stakeholders therein. In some embodiments, the PPE may be calibrated and configured to, at least in part, determine a boundary or perimeter representing the reach and extremities of the PUM, so as to, at least in part, determine the likelihood of an interaction of the PUM with an external object or person. This interaction can include, for example, a PUM traversing a SEE, and the potential intentional or unintentional interactions with the furniture, fittings or other objects located in the SEE.

In some embodiments, one or more digital twins may be employed, at least in part, with a PPE and one or more AI/ML system to generate representations of the PUM's movements in a SEE, based at least in part, on the one or more patterns or behaviors of that PUM, so as to identify potential situations that may result in adverse health, care, safety or wellness outcomes for the PUM. In this manner, one or more corrective or mitigating actions may be evaluated using the digital twin, PPE or AI/ML systems, which can be communicated to the PUM, other stakeholders, for example a carer or to other systems.

One aspect of the PPE may be the degree of certainty of an action of the representation of the PUM, where for example one or more AI/ML system is operating, and the PPE is, at least in part, enabling such AI/ML system to operate as a RAG, where the PPE enforces the relative laws of physics of movement of the PUM, based for example on appropriate sensor, device or system data patterns, mobility modules, mobility frameworks in any arrangement including the one or more data sets generated by the one or more sensors, devices or systems that populate such representations.

One or more sensors, devices or systems may also use positive confirmation of an action of a PUM. For example if the PUM is deemed to be sitting based on the data sets generated by the one or more sensors, device or systems and the framework representing the PUM, a query in the form of a question may be communicated to the PUM, to confirm the current mobility state of the PUM. For example, if the system determines that the state of the PUM is a sitting state, a query, (e.g., a message on a smart phone, Personal Emergency Response System (PERS) or other worn, carried or implanted device or a speaker of audio device associated with such a device or deployed in the SEE) may be conveyed to the PUM asking for confirmation of the current state of the PUM, which may be a sitting or some other actual state.

In comparison to the many existing physics engines employed in games, computational fluid dynamics, robotics and other representations of the physics governing the movement and responses of real-world systems, the PPE is adaptive to the specifics of a particular PUM, such that the PUM movement characteristics, including the behaviors of the PUM, can be aligned to the physical capabilities of that specific PUM. This alignment includes the PPE correlating the physics of the PUM as that PUM changes over time, for example as the PUM ages or after the PUM have had a medical procedure, such as hip or knee replacement or other mobility impacting event. This adaptive capability in combination with snapshots or other persistent representations of the mobility state of the PUM, supports the PPE in predicting the likely capabilities of a PUM, though for example using one or more AI/ML systems or digital twins. There may also be a continuous alignment with the PUM state, for example when the calibration of the PPE is being undertaken or when the health, care, safety or wellness state of the PUM is undergoing rapid change.

For example, when a PUM condition changes, such as experiencing the flu or other common ailment that can impact the mobility of the PUM, the PPE is realigned based on the updated condition of the PUM. These ailments may be declared by the PUM or other stakeholders, or may be determined through observation, for example through the one or more sensors, devices or systems of the SEE, where the mobility behaviors are outside the one or more thresholds of the PUM mobility behaviors (e.g., the quiescent behaviors). In this example, the PPE may invoke a messaging system to ask the PUM or other stakeholders whether the PUM is experiencing an ailment. In the example of arthritis, a knee replacement procedure and the like, these changes may be permanent rather than temporary. This alignment can include adjusting movements for differing locations, such as gardens, slippery floors and the like. For example, after spilling a drink in a kitchen, the PUM may consciously shift to a more cautious movement pattern to avoid slipping and falling.

In some embodiments, the PPE may also include any detectable movements representing, at least in part potential side effects of the one or more medications that a PUM is taking, where for example the eyesight of the PUM is impeded, the PUM has a loss of balance, experiences vertigo and the like, where for example the PUM uses the walls, furniture or fittings of the SEE to steady themselves.

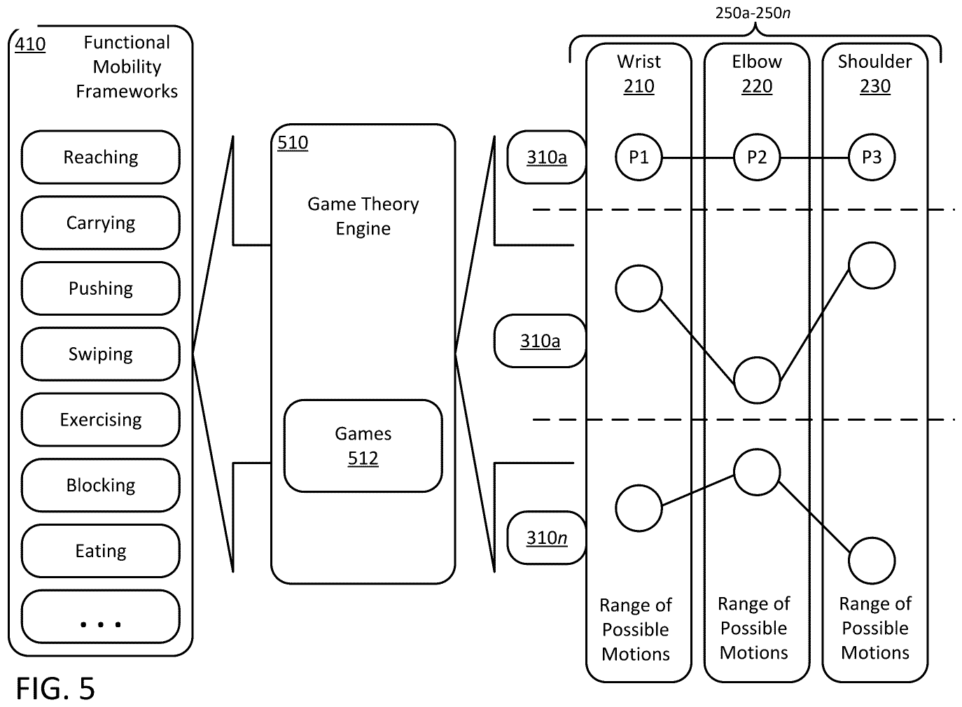

FIG. 5 illustrates an example of movement of a set of joints 250 (e.g., a wrist 210, elbow 220, and shoulder 230), which in various orientations are represented by mobility modules 310. These modules 310 may be evaluated by one or more game theory engines 510 configured with a set of games 512 representing the potential arrangement of such movement modules 310. These arrangements may have outcomes that are represented by one or more mobility frameworks 410.

In some embodiments, the interactions of the SEE may be incorporated into the representation of the PUM. For example, if the SEE includes one or more haptic sensors attached, for example, to the floor, the footfall of the PUM may be measured as the PUM moves. In this manner, the typical movement footfall may be calculated and one or more thresholds determined, such that if sensor readings of the footfalls exceed these thresholds (e.g., for noise, force applied, speed between successive readings, etc.), this exceeding of the thresholds may represent, for example, a stumble or fall, which could indicate a detrimental health, care, safety or wellness event.

In some embodiments, each joint may be considered as the base of a cone of height (N), being the distance from the base to the next joint or the extremity, for example fingers, and radius (R) being the length of the limb attached to the joint. These cones can be combined, such as an arm where there are three joints (e.g., shoulder, elbow and wrist), each of which has a specific range of motion. In this manner, a cone of possible motions, including reach may be defined. This approach may form part of a mobility module or mobility framework.

Many movements of the human body involve multiple joints operating in a particular arrangement involving both parallel and sequential operations. For example, typing on a keyboard can involve wrists, elbows, shoulders and neck, where each of these joints operates in response to the fingers activating the keys selected by their owner. Many of these movements can be subtle, such as a shoulder movement when typing. In many human movement monitoring systems and those intended for robotics or other moving systems, a high degree of accuracy can be required. However, for the monitoring of the health, care, safety or well-being of a PUM, the objective is to identify when a movement does not align with the typical movements or behaviors of the PUM. This approach can be used to reduce the degree of accuracy required from any one or more sensors, devices or systems of a SEE, such that a best fit approach may be employed.

In some embodiments, one or more game theory games may be deployed to, at least in part, identify the movement of a PUM, where the game can involve a number of joints, for example as players, where the moves of these player-joints can represent specific actions of those joints and to identify potential outcomes. This game deployment can include one or more multistep games where sequences of joint movements are combined. In some embodiments, the personal physics engine may include the rules of human dynamics, including body muscular/skeletal mechanics, body mechanics, generalized physics parameters or physics ratios and relationships and sets of one or more games representing the potential movements of those dynamics, which can then be represented as behaviors of a PUM. These behaviors can be aligned with the specifics of a particular PUM.

### TRANSFORMER FOR MOBILITY

In a similar manner to the use of transformers for LLMs, transformers for mobility may be used in the context of representing, at least in part, a PUM's mobility. These transformers, in common with transformers for LLMs use positional encoding to determine the sequence of a particular input in a sequence of inputs. In some embodiments, the one or more sensors, devices or systems present in a SEE can generate data sets that can be formed into sequences or patterns. For example, these patterns may include PUM activities, such as walking, sitting, lying or the like. In some embodiments these patterns can be in the form of tokens, which may provide various levels of processing to the patterns that are configured for ingestion by one or more AI/ML models or other systems.

In some embodiments, one or more patterns may include a mobility module, which is the representation of, at least in part a movement of a PUM. For example, this may include combinations of PUM joints and limbs, such as reaching for an object, carrying a plate, extending a hand, walking and the like. There can be mobility modules that are determined to be impactful on the health, care, safety or wellbeing of the PUM, such as for example, rapidly pushing one or both hands forward, for example to stop a fall or to grab a wall or object for balance.

In some embodiments, sets of mobility modules can form, at least in part, a mobility framework which is a representation one or more movements of a PUM. For example, a mobility framework can comprise movements of an arm and hand, such as when picking up or holding an object, moving of the legs in a specific manner such as walking, rising from sitting and sitting from standing, and the like. Each of these mobility frameworks includes representations of the measurements by the one or more sensors, devices or systems present in the SEE of a PUM undertaking these movements. These mobility frameworks can form sequences that represent the movements of a PUM from, for example from an origin to a destination.

Mobility frameworks or mobility modules may be represented as tokens, and as such these tokens can be formed into sequences thereof. In some embodiments, a mobility framework represented as a token can have a relationship of preceding tokens, also representing mobility frameworks or peer tokens, representing further mobility frameworks, but having no relationship with other tokens, such as those that are created or generated after the time-period of the preceding or peer tokens.

In some embodiments, input encodings based, at least in part, on patterns generated by one or more sensors, devices or systems present in a SEE may be used as part of one or more positional encoding matrices. These patterns may include one or more data sets from the one or more sensors, devices or systems that are present in a SEE. For example, one or more haptic sensors, may generate data sets that represent the footfall of a PUM, where each leg may generate a footfall and a sequence of such footfalls can represent a PUM walking. This pattern may form at least in part, in some embodiments, a mobility module, which can form part of a mobility framework. For example, the mobility framework may include mobility modules representing patterns generated by one or more haptic, audio or other sensors and one or more images representing the gait of the PUM, generated, at least in part by one or more sensors, devices or systems, including for example cameras.

In a typical LLM, transformers may be employed to create a set of relationships between the elements forming the sequence of data sets that the transformer is operating upon. These relationships can include metrics, including relevancy, risk, and the like. A similar approach may be employed when considering the mobility of a PUM in a SEE, where the transformer operates on the elements forming a sequence of data representing their movements. In an example embodiment, the data sequence can include, in any arrangement, but is not limited to: data sets generated by the one or more sensors, devices or systems present in a SEE; patterns based on such data sets; mobility modules including one or more data sets or patterns; mobility frameworks including one or more mobility modules, patterns or data sets. These inputs to the transformer may undergo positional encoding, where for example, data sets, patterns, mobility modules or mobility frameworks and the like can be represented by one or more tokens, where such tokens can include vectors representing a 3D space, and can form an N dimensional space.

In some embodiments, the vector data that forms part of a pattern, mobility module or mobility framework can include for example, direction, velocity or acceleration. These data can be included in the token representing such movement. In many typical LLM transformers, the output of the positional encoding is fed to a multi-attention module, which in many cases accepts linear input and uses a dot product approach to generate outputs. In a 3D vector environment, where 3D direction, velocity and acceleration may be evaluated by a multi-attention module, these modules may incorporate one or more topological representations of these tokenized inputs and employ, for example, manifolds, such as Reimann manifolds, or other topologies such as Hilbert spaces. One aspect of this approach may be the deployment of non-linear representations, including the use of an N dimensional matrix for representation of the relationships between the one or more tokens forming the input sequences.

In some embodiments, one or more input tokens, representing data sets, patterns, mobility modules or mobility frameworks may be masked, such that a transforming function such as one or more machine learning models or algorithms may then predict these masked tokens in the context of incoming token sequences. This approach can be employed as part of a data set, pattern, mobility module or mobility framework validation process. This approach can inform as to the physical feasibility of the token sequence, based at least in part, on one or more PPE.

When generating physics simulations as part of a PPE or when evaluating the current state and movement patterns of a person under monitoring (PUM), the inclusion of one or more previous states or recent movements can impact the accuracy of such simulations or evaluations. When AI/ML mechanisms are deployed by a PPE, previous state inclusion can be achieved by using techniques such as recursive neural networks (RNN) and attention mechanisms, such as transformers. This approach can include attention mechanisms that help neural networks focus on different parts of an input sequence when making predictions, improving their ability to capture dependencies and contextual relationships within the data. This approach allows models to weigh inputs differently, concentrating more importance or “attention” on certain elements while processing sequences. In some embodiments, the attention of a PUM to the PUM's environment, an event, a situation or other stimuli may inform the AI/ML attention mechanisms on those particular elements.

Unlike feedforward neural networks, RNNs have connections that form directed cycles, allowing RNNs to maintain internal state and process sequences of inputs over time. This feature makes RNNs particularly useful for tasks where the sequence or context matters, such as language modeling, speech recognition, and time-series prediction for processes such as sequences of movements, including those of a PUM.

Attention mechanisms in machine learning are techniques that allow AI/ML systems to focus on specific parts of the input when performing a task, similar to how humans pay selective attention. Attention mechanisms provide a means for models to “attend” to certain parts of the data more than others when making predictions or generating outputs, leading to improved performance on various complex tasks that involve sequences of data and require understanding of context. This approach is particularly useful in sequence-to-sequence tasks like language translation or speech recognition, where capturing dependencies regardless of the position of words in the sequence can be important. Accordingly, attention mechanism can be applied to sequences of movements, for example those represented by patterns, mobility frameworks or mobility modules, that form part of a PUM's behavior patterns.

One purpose of attention mechanisms may be to compute context vectors that dynamically weight different input features based on the relevance of those input features for a specific output element. For instance, in natural language processing (NLP), when translating a sentence from one language to another, an attention mechanism can help identify which words or phrases are most important at each step of the translation process. Similarly, when evaluating a PUM's sequence of movements, for example, movements that take the PUM from a sitting position to a bookshelf nearby to grab a book, each individual movement (e.g., standing up, which can be represented by a mobility framework) or sequence of movements (e.g., five steps from the chair to the bookshelf, with two changes in direction, which can be represented by a set of mobility modules or mobility frameworks) can be correlated, based on the order, location, and relationship between the mobility frameworks or mobility modules thereof, and be represented as vector of one or more values for each respective movement or sequence thereof, to inform, calibrate or configure one or more contextual interpretations of these mobility actions, including sets thereof, representing, at least in part the intentions of a PUM or other stakeholders in a SEE, which may form part of the PPE.

Vectors may be employed for positional embedding, for example rotary positional embedding (RoPe) for 2D vectors, which can be extended to 3D vector representations.

### INTENTIONS

A PUM domiciled in a SEE can exhibit a set of behaviors that include the movements or mobility of the PUM. These behavior sets can be identified, at least in part by the one or more sensors, devices or systems deployed or present in a SEE, including when evaluated by one or more care hub or care processing system, including those incorporating an AI/ML system, such as an LLM/LCM or a PPE.

In some embodiments, a PPE may include patterns, mobility modules or mobility frameworks that are representations of the these sets of behaviors and movements, where the elements or modules of these frameworks can represent the intentions of the PUM. For example, a PUM may intend to reach for a book on a shelf, and in so doing may move towards the shelf so that the PUM can reach that book, which can be observed by the one or more sensors, devices or systems present in the SEE, which generate data sets that can be represented as a set of movement modules, such as stretching an arm, and then gripping the book, which in turn can be represented by a mobility framework.

For example, a mobility framework may include a set of mobility modules that include the one or more joints of a limb. Another example may involve the orientation of the head, such that a mobility framework, for example, “reach”, can include the mobility module of the limb and the neck.

In some embodiments, a set of mobility frameworks representing physical characteristics of a PUM, may include one or more walking frameworks, for example, striding (representing walking behavior such as on a hike), ambling (representing walking behavior such as in a garden or whilst shopping), internal (such as when moving inside a domiciled environment) or the like. Each of these frameworks may have one or more relationships with one or more intentions, which in some embodiments can be instantiated as an attribute or characteristic of the framework. For example, an intention may be seeking, where the PUM is intending to obtain an object or an outcome. For example, this intention could be seeking to obtain the TV remote, a book from a shelf, rescuing the knitting from a cat, and the like.

In some embodiments, a set of intentions can be represented as characteristics of behaviors, in that when a PUM is engaged in one or more movements, there is at least one reason or intention for that movement, even if the PUM is forgetful, for example going to the kitchen and forgetting why.

Intentions can be expressed, in some embodiments for example, as triples (or other tuples), with an initiation, action, and result. For example, an initiation may be rising from a sitting position, an action may be traversing from the sitting location to another location, and a result may be making a cup of tea or coffee at the second location. Each of these elements of an intention can be represented by one or more data sets from one or more sensors, devices or systems that form part of a SEE, where for example, each of these elements, in whole or in part, forms or matches a pattern representing those behaviors.

In some embodiments, the initiation may indicate a range of potential intentions. For example, rising from sitting could indicate, going to a destination, which may be a focal or attention point, for example kitchen or bathroom, reaching for a TV remote, book or electronic tablet or simply stretching. In some embodiments, the set of behaviors that can represent the activities of the PUM may have common initiations and as such the one or more care hub or care processing systems may, for example, employ a PPE to evaluate a set of potential behaviors. This deployment can include the PPE interacting with one or more game theory modules or one or more AI/ML systems to predict, at least in part, the intention of the PUM. As the actions performed by the PUM unfold, the reason for the movement of the PUM can be become more discernable, based at least in part on the one or more data sets or patterns of the one or more sensors, devices or systems where the PPE in conjunction with the game theory or AI/ML systems can provide the earliest recognition of the intention of the PUM.

One aspect of the PPE may be the identification of intentions which have no result or an unanticipated result. For example, the PPE may form part of a monitoring system that identifies and tracks the behaviors of a PUM, where the PUM initiates a movement, for example rising from sitting to standing, which the PPE recognizes as a pattern that could be an initialization of an intent. The PPE may then load a set of frameworks, including mobility frameworks, representing a set of intentions, for example traversing to the kitchen, bathroom, bedroom, garden, door or other location available to the PUM. Each of these frameworks may have multiple possible actions or results, which in some embodiments may be represented in the form of a graph database or other arrangement. In some embodiments, a PPE may employ game theory, in whole or in part, to select which of the possible actions is the PUM intends to undertake. As the PUM traverses the SEE to a location, the PPE may, using the data sets generated by the one or more sensors, devices or systems of the SEE, populate one or more frameworks, to identify the particular behavior of the PUM and update the intent of the PUM. For example, the PPE may initially be identified with intents to move to the bedroom or the kitchen, which are on opposing sides of the SEE, and the PPE may remove an intent to move towards one of the locations based on a direction of travel towards/away from a respective location. Additionally or alternatively, when the PUM has arrived at the location, for example entering another room or space, the monitoring systems including a PPE can further refine the selection of the potential intentions of the PUM and evaluate the SEE data to match that intention. For example, identified intentions of “retrieve an object”, “go to bed”, “get something to eat” and “get something to drink” may be reduced to “go to bed” and “retrieve an object” when the PUM is identified as entering a bedroom (rather than a kitchen) or may be reduced to “retrieve an object”, “get something to eat” and “get something to drink” when entering a kitchen (rather than a bedroom).

In this manner, the monitoring systems and PPE can also identify when the PUM has not completed the intention, for example entering the kitchen and then not undertaking any kitchen based behavior, such as making tea, coffee, preparing food or searching the fridge or the like. For example, after identifying the intention of the PUM and identifying that the PUM entered a zone associated with an intention and not observing performance of actions associated with the intention (or an alternative potential intention) before leaving the associated zone, the PPE can identify that the intention was not completed.

The use of context, behaviors or location can form part of determining the intention, which can be explicitly expressed by the PUM, of the PUM as the PUM exhibits one or more movements. In some embodiments, the identification or determination of intention can include the identification or mapping of one or more locations within a SEE, which can be defined as focus or attention points.

In some embodiments, a PPE may maintain a repository of sensor data patterns, mobility modules or mobility frameworks which are representations created at least in part by one or more AI/ML systems that have been trained on data sets of multiple PUM.

A PPE may be integrated with one or more Internet of Things (IOT) devices, such as, for example, refrigerators, scales, ovens, other cooking devices, water flow measuring devices, electricity or gas measuring devices, motion detectors, light detectors and the like. The data sets from these IOT devices may inform and be used to identify one or more behaviors of the one or more PUMs that are represented by a PPE.

For example, electricity usage may be used, at least in part, to calculate the heating or cooling that a PUM is experiencing.

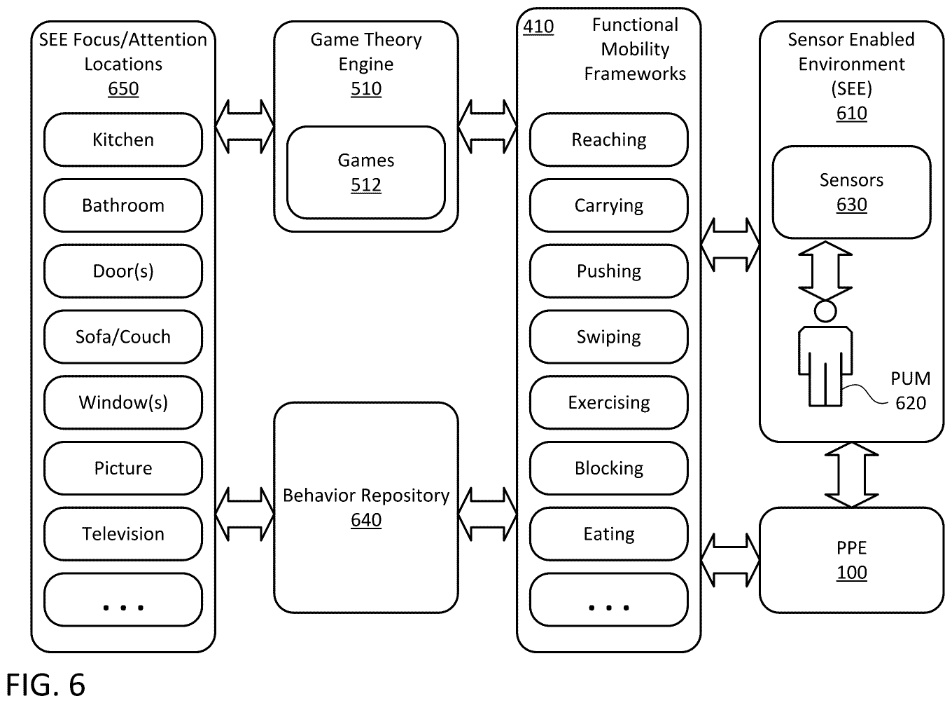

FIG. 6 illustrates an example of a SEE 610 where a PUM 620 is monitored by one or more sensors, devices or systems present in the SEE (generally, sensors 630), including those that are worn, implanted or carried by the PUM 620, those that are associated with or deployed in the SEE 610 (e.g., any sensor, device or system not worn, implanted or carried by the PUM 620 provided for monitoring the PUM 620, the SEE 610 or individuals therein), and those that are communicated to the SEE 610 from an external source (e.g., a temperature sensor or weather reporting device for conditions outside of the SEE 610). The SEE 610 includes or is in communication with one or more PPE 100 representing the PUM-personalized physics and consequent mobility characteristics. These PUM mobility characteristics may be aligned with one or more mobility frameworks 410. The behavior repository 640 may include one or more mobility frameworks 410 representing the PUM behavior characteristics, which in turn may be aligned with the SEE focus or attention locations 650. In some embodiments, one or more game theory engines 510 and games 512 may be employed to evaluate mobility frameworks 410 or behavior characteristics to, at least in part, identify attention or focus locations 650, including the one or more strategies employed by a PUM.

### INTENTION AND ATTENTION

In some embodiments, the one or more sensors, devices or systems present in a SEE can generate data sets that can be evaluated to, at least in part, determine the attention of the PUM at a specific time. For example, gaze tracking and similar approaches can be employed, through the use of one or more cameras or image sensors, to identify the direction in which a PUM is looking.

The PPE can include one or more representations of the mobility of the body's limbs, including the neck and head of the PUM, such that the direction of the gaze of the PUM can be determined. This representation may be reinforced by the natural habits of the PUM, where certain characteristics are exhibited as behaviors on a repeated basis, such as craning the neck forward, angling the chin down, scanning an area, looking towards or away from certain area in the SEE (e.g., towards a window, television, entryway, picture, or location of another PUM or animal in the environment versus away from a corner, mirror, light fixture, etc.), jerking the head in a direction or the like.

In some embodiments, these movements may form part of one or more behaviors, where for example before opening a cupboard, the PUM looks up at the cupboard, or before traversing a set of stairs the PUM looks down at the first stair.

These movements and actions can, at least in part, provide one or more data sets, for example patterns, behaviors or data sets generated by the one or more sensors, devices or systems in a SEE.

In some embodiments, one or more sensors may detect an event, for example a noise, impact, image or other occurrence that can trigger a response of the PUM through moving of a part of the PUM's body, for example turning a head towards the source of the event. This event-movement correlation can represent the triggering of the PUM's attention, which, in some embodiments, may involve the configuration of one or more sensors, devices or systems to generate data sets in response to the PUM's movement, which at least in part, can be used to determine the potential for such an event to be detrimental to the health and well-being of the PUM.

In some embodiments, PUM sensing can be expressed in terms of the ability of the PUM to sense, that is the sensors of hearing, tactile, vision, taste and smell of the PUM, each of which can have one or more metrics.

One aspect of an event driven attention focus may be the PUM's ability to recognize an event. For example, if the PUM is hard of hearing and does not have a hearing device inserted, the PUM may not recognize the sound of an event, for example an object falling on the floor.

However, a PPE may be configured to represent such a physical constraint of the PUM as part of a care hub or care processing system, where one or more sensors, devices or systems have been triggered by such an event, for example a microphone, haptic sensor or camera, these one or more sensors may generate an event, which may then be communicated to the PPE, such that the PPE can represent the PUM's response, or in this example lack thereof, and as such the one or more care hub or care processing system may generate an alert, for example in the form of a spoken or visual message to the PUM, a haptic indication to the PUM or another form of alert, for example using a smart television, smart watch, smart phone, personal monitor or the like, such that the PUM is made aware of the event. This alert can include alerting other stakeholders such as carers, neighbors or other stakeholders present in or having access to the PUM in addition to alternatively to alerting the PUM.

In one or more SEEs, various locations within the SEE may be identified or classified as focal points, in that a PUM may at various times focus intention or attention on such points. Some examples, include functional aspects of the SEE, such as kitchen, bathroom, doors, windows and the like. The kitchen may include multiple focal points, such as refrigerator, sink, cooker, coffee maker and the like. Similarly, the bathroom may include bath, shower, toilet, sink, mirror and the like as focal points.

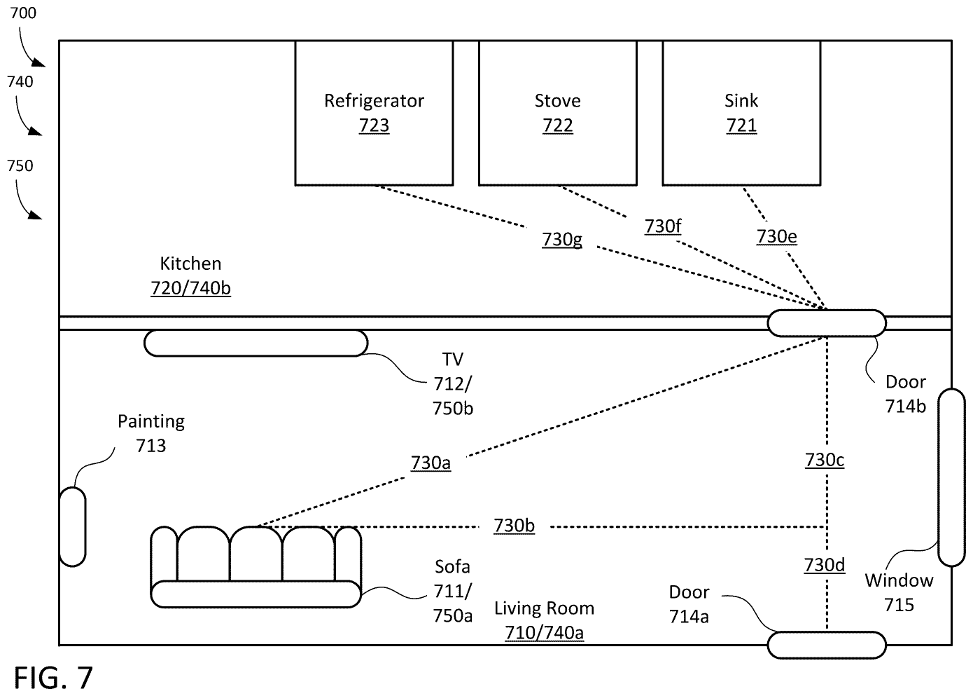

FIG. 7 illustrates an example of a SEE 700 with two example areas, a living room 710 and a kitchen 720 separated by a wall, with a connecting door 714b (generally or collectively, door 714) for access. In this illustrative example, the various focal points in the environment include a sofa 711, an external entry/exit door 714a, a window 715, a painting 713, a television (TV) 712, and the connecting door 714b, all of which are in the living room 710. The kitchen 720 includes a refrigerator 723, stove 722, and sink 721, which are identified as focal points within the kitchen 720. Although illustrated in FIG. 7 with a given number of areas 740a-n (generally of collectively areas 740 of the living room 710/740a and kitchen 720/740b) and a given number of focal points 750a-n (generally or collectively, focal points 750; e.g., the sofa 711/750a, the TV 712/750b, etc.), the present disclosure contemplates that more, fewer, or different areas 740 and focal points 750 may exist in various SEEs 700. Additionally, some focal points 750 may be modeled as belonging to more than one area 740, such as the connecting door 714b, which may be modeled as a focal point 750 for the living room 710 and for the kitchen 720. In the illustrated example, the potential paths 730a-g (generally or collectively, paths 730) for a PUM who is located, for example, on the sofa 711 to move to one of these focal points 750 is exemplified by the various individual example behavior paths 730.

In some embodiments, these focal points 750 may be mapped as part of the initialization, calibration or configuration of the SEE 700, including the one or more sensors, devices or systems present therein, and can have specific sensors, devices or systems monitoring those locations or the interactions with those locations of the PUM or other stakeholders. These focal points 750 may form part of the behaviors of the PUM, for example as origins or destinations for the movements of the PUM.

For example, a SEE may include a set of locations or objects of interest or locations or objects of interaction, where the PUM determines the interaction, which can include windows (which may be opened/closed/looked through), doors (which may have sensors indicating a state (open/closed/in motion)). Similarly, the PUM may not have a great deal of focus on a wall, although if the wall has a picture or other feature, this feature may be mapped as an object of interaction or focus. This identification of locations or objects of interest can include recognition or mapping of the contents of a SEE, which may include those elements that are permanent fixtures of the SEE and those that are temporary or both (e.g., a television or electronic picture frame being permanently located in a given location, but having different images displayed thereon at different times or a piano or radio being permanently located in a given location, but having different audio output at different times).

In some embodiments, there may be various behaviors that are appropriate, in that the behaviors form part of the behaviors of the PUM, and can form part of a quiescent state, for the interactions with locations or objects within the SEE. In various embodiments, the quiescent state represents the behaviors and movement frameworks that do not cause the system to generate an alert or other follow-up action (e.g., an actionable state). For example, a behavior of a PUM sitting down to watch TV at three in the afternoon may be considered a quiescent state for the PUM whereas sitting down to watch TV at three in the morning may not be considered a quiescent state for the PUM based on the observed wakefulness patterns for the PUM (e.g., indicative of a disrupted sleep pattern). For example, a behavior of a PUM vigorously using a stationary bicycle at nine in the morning may be considered a quiescent state for the PUM whereas the PUM lackadaisically using a stationary bicycle at nine in the morning may not be considered a quiescent state for the PUM based on the observed exercise patterns for the PUM (e.g., indicative of poor health preventing vigorous exercise). For example, a behavior of a PUM more noisily or forceful impacting a surface than a threshold deviation from previous noises or forces of impact with that surface may be indicative of a fall or arrested fall, which the system may treat as a non-quiescent behavior for which follow-up actions are initiated.

One aspect of these focal points may be the use of gaze or eye tracking or other metrics of focal awareness, for example, time, to align such focus metrics with the focal points. In this manner, the intention or attention of a PUM may be calculated, in whole or in part. The PPE may then provide potential movements of the PUM should the PUM undertake one or more movements in response to the attentions of the PUM. For example, if an attention focus of a PUM is directed to a kitchen appliance, the PPE may provide sets of data, for example, as movement frameworks, that represent the anticipated movements of the PUM. These data can then be evaluated by the one or more care hub or care processing systems, for example, those systems that are receiving the one or more data sets or patterns generated by the one or more sensors, devices or systems present in the SEE.

In some embodiments, the distances within a SEE may be known or calculated and as such form a metric, for example, if PUM focus is on a distant (for example, where distant is outside reach of PUM from a current location) location or object (for example a door), the PPE may be employed to estimate the movements of the PUM from the current location to the location or object of focus of the PUM. The PPE may then form part of an evaluation that can determine, at least in part, if there are any potential risks to the PUM in undertaking this activity. This evaluation can include providing the PUM or another stakeholder with one or more messages, such as, for example, reminding the PUM to stand up slowly or informing the carer that the PUM may need assistance, which can include providing such messages to one or more robotic, autonomous or other supporting systems.

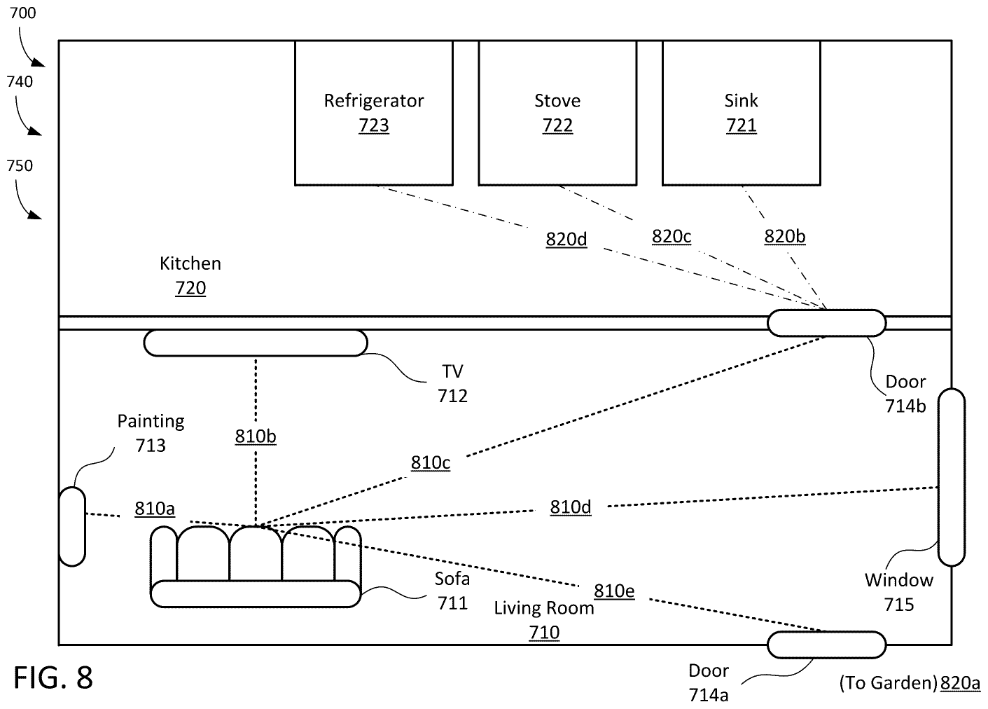

FIG. 8 illustrates an example of a SEE 700 including two areas 740a-b, a living room 710 and a kitchen 720. For a PUM situated, for example, on the sofa 711, the attention of the PUM may be directed to any one or more focal points 750, for example, a painting 713, a window 715, a TV 712, an entry exit door 714a or a connecting door 714b, where the attention of the PUM is represented by one or more attention paths 810a-n (generally or collectively, attention path 810). In this example, the use of one or more sensors, devices or systems to determine the current attention path 810 of the PUM can indicate the intention of that PUM to undertake movement towards such focal points, for example, the doors 714 or window 715, whereas if the focal point is the TV 712 or painting 713 the intention of the PUM may not include any movement. Additionally, when the attention path 810 of the PUM is located to a door 714 or other pathway out of the current area 740, one or more intention paths 820a-n to focal points 750 outside of the current area 740 (e.g., a first intention path 820a to a garden accessible via the door 714a or viewable through the window 715, a second intention path 820b to a sink 721 in a kitchen 720).

For example, in some embodiments, a PUM may be located at an origin point, which can be represented as “Origin=location L1”. These origin points may be focal or attention points or may reference such focal or attention points. The PUM may have a movement metric, represented for example by velocity and acceleration of (N). These movements can include some variation for example, crossing of a PUM's legs which is a movement of a limb, though not involving mobility. In this example there may be a threshold where if movement is less than (N), then there is no mobility. Accordingly, the systems described herein may differentiate between ambulatory movement (e.g., walking, running, wheeling, being pushed), incidental or stationary movement (e.g., scratching an itch, blowing a nose, crossing legs, shifting weight, turning a head), false-start or deflected movement (e.g., a movement to rise from sitting to standing that did not result in standing, a movement to lower from standing to sitting that returned to standing within a given time period, movement to travel from point A to point B that resulted in premature return to point A or alternative movement to point C) and various other categorizations, which may be treated differently in the PPE and the SEE. For example, if the PUM is identified as having false-start movement, such as apparent difficulty in rising from a sofa, an alert may be generated for a caretaker to assist the PUM in reaching a location of interest or to retrieve an object of interest, or to confirm that the PUM is trying to get up from the sofa versus reposition on the sofa to become more comfortable.

In this example, there may be a focal or attention location, which may be described as a destination or target, represented by for example, as Location L2. There may, for example, be a number of potential targets, determined at least in part by the one or more focal or attention points, that are calculated by, for example, monitoring of the PUM gaze or head movement. In this example, if the movement of one or more metrics exceed a threshold, representing mobility of the PUM, the one or more care hub or care processing may use a PPE or one or more AI/ML systems to determine the likely targets for the PUM. This determining can include the use of one or more game theory games to, at least in part, filter the possible targets.

In some embodiments, the determination of movement may include the recognition that the PUM has certain involuntary movements, for example where a hand shakes, due at least in part to a health condition, current stress level or other permanent or transitory factors affecting the PUM. These movements are described herein as jitter, where one or more limb or extremity of the PUM, for example a hand or foot, has a certain amount of motion. This jitter may form part of the threshold used to determine whether the PUM is initiating a mobility activity and as such may be considered as part of the overall monitoring of the PUM by, for example, a care hub or care processing system and may be used as part of the configuration of a PPE, which can be personalized to that individual PUM. Jitter, which may include rest tremor and action tremor, will be understood by those of skill in the art to describe an involuntary shaking, trembling or oscillation of a body part (often rhythmically), which may occur within a defined range of motion or an entire range of motion for one or more limbs, and generally affects fine motor control to a greater extent than gross motor control (cf., spasms). In various embodiments, jitter may be understood to include other movements that are initiated involuntarily by the PUM, such as minor movement of limbs due to inhalation/exhalation, actions taken while sleeping, blinking, nasal flaring, etc., and may include soft-tissue or jointless motions. Accordingly, the system may model the PUM via various models with various numbers of joints (including no joints) and account for movements that occur without a corresponding joint (or with no joints) and various voluntary and involuntary movements thereof.

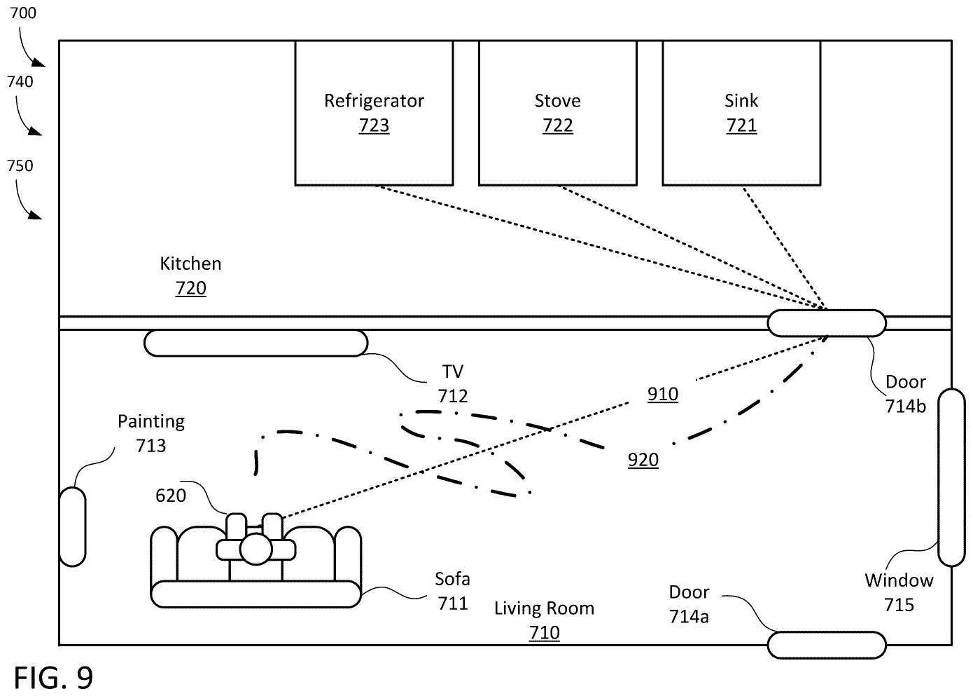

FIG. 9 illustrates an example of a PUM 620 who is located at a sofa 711, which is in a SEE 700 including two areas 740a-b (a living room 710 and a kitchen 720), who has a set of behavior paths 910 which represent movements of the PUM 620 in the SEE 700 on a regular basis. In this example, the PUM 620 moves from the sofa 711 towards the connecting door 714b, which provides access to the kitchen 720. However, in this example, the PUM 620 may start moving in one direction, for example towards the TV 712, before heading towards the window 715, then again towards the TV 712, and then again towards the window 715, before finally reaching the connecting door 714b. This movement path 920 may indicate that the PUM 620 is having difficulty with mobility or may have difficulty with intention or attention as the PUM 620 navigates the SEE 700. In some embodiments, the behavior paths 910 may have one or more thresholds where the movements of the PUM 620, although not the most direct path, may still form part of that behavior.

One aspect of this approach may be the use of vectors to correlate the intention of the PUM with the mobility actions of the PUM. For example, if the PUM turns a gaze to a recognized focal or attention point, for example, a kitchen, and then undertakes a series of movements, which may be represented by a series of movement frameworks, the PPE may be configured to represent the optimum, expected or typical path from the initial location of the PUM to the identified target location. These movement paths may then be compared with the actual path the PUM is taking to identify, at least in part, the mobility capabilities of the PUM, or to generate one or more messages to the PUM, other stakeholders or to one or more suitably equipped appliances, IoT devices or other systems that the PUM may engage with. In some embodiments, the PPE based determination of the movement vectors of the PUM can initiate one or more commands to, for example, devices or systems that can accept such commands, for example, turning on an oven, kettle or other device.

In some embodiments, there may be one or more thresholds for the vectors used in determining the possible targets of the PUM intentions or attentions, where, for example, a set of cones, representing the potential reach of the PUM form an initial point, may be used, at least in part, to represent the potential movements vectors of the PUM. These potential movement vectors may then be compared with the focus or attention points that are mapped in the SEE, so as to reduce the possible set of targets and enhance earlier detection of the intention or attention of the PUM. For example, this vector variance may include one or more thresholds for vectors, through, for example, representations including cones for determination, may be generated or used by one or more AI/ML systems in conjunction with a PPE to, at least in part, enable matching of these vectors to focal or attention points, which can be included in behavior representations, including for example as tokens.

In some embodiments, fluid neural networks may be invoked to ascertain the combinations of patterns, movement modules, movement frameworks, intentions, attention, behaviors, locations, focus or attention points based at least in part on the PPE of a PUM.

In some embodiments, one or more timers may be employed to, at least in part, determine the time period for one or more PUM movements, including movement modules, movement frameworks or sequences of movements including these. Timers may also be employed to measure limb jitter or other movement affectations.

In some embodiments, one or more AI/ML system may be trained on such timing data so as to determine, at least in part, the typical period for one or more movements or sequences thereof. This determination can include, for example, minimum and maximum periods, which can be used to form thresholds for such movements, such that one or more care hub or care processing systems may evaluate such movement time periods to, for example, create alerts, events or messages if, for example, one or more thresholds are exceeded. Such timing thresholds can be based on or referenced to one or more locations, for example focus or attention points or one or more objects, including for example appliances, IoT devices or the like.

The use of vectors, timing or movement metrics combined with patterns, movement modules or movement frameworks supports the rapid and effective determination of the PUM intentions or attention in regard of mobility of the PUM within the SEE. This rapid and effective determination can provide stakeholders, including the PUM, with messages and communications so as to the ease or enable the quality of life of the PUM to be enhanced. In some embodiments, one or more timing thresholds may be employed to, at least in part, determine if a movement, including a movement module or movement framework is being initiated. This employment can enable the identification of these movements in light of, for example, a PUM having shakes or jitter in a limb or joint. This determination may be particularly the case where the condition of the PUM includes arthritis or immune systems deficiencies such that the PUM may need to make more than one attempt to undertake a movement module, such as gripping a glass, turning a tap or similar. The timing thresholds may be employed for the identification of such movements or, if these thresholds are exceeded, to generate one or more messages or alerts that can be communicated to the PUM, other stakeholders, care hubs or care processing systems.

In some embodiments, the measuring of jitter by, for example, one or more sensors, devices or systems present in the SEE, can provide data sets indicating the trajectory of any debilitation of the capabilities of the PUM, which can then inform the PUM, other stakeholders or care hubs or care processing systems, which can then employ appropriate care and wellness strategies in response. These trajectories may be calculated using, for example, a PPE in collaboration with an AI/ML system.

In some embodiments, one or more care hub or care processing systems may be employed to aid or guide the attention of the PUM to an anticipated, scheduled, responsive or other behavior, where that behavior includes one or more mobility actions, including for example initiating one or more movement modules, movement frameworks and the like. For example, this can include one or more communications to a PUM, stakeholders or other systems, including for example worn, implanted or carried devices that are in the presence of the PUM.

In some embodiments, time may be used as a metric as part of the monitoring systems and can be, in some embodiments, quantized in a manner that is matched to the behaviors of the PUM. For example, the quantization of time may be such that certain sensors, devices or systems may be employed to observe the real time PUM behaviors, such as for example when resting, reading, watching TV or sleeping. These observations can enable the other sensors, devices or systems to conserve resources, such as battery or other power consumption, reduce messages or other communications, reduce processing requirements, for example using cloud, hub or other system processing power, reduce heat generation or the like.

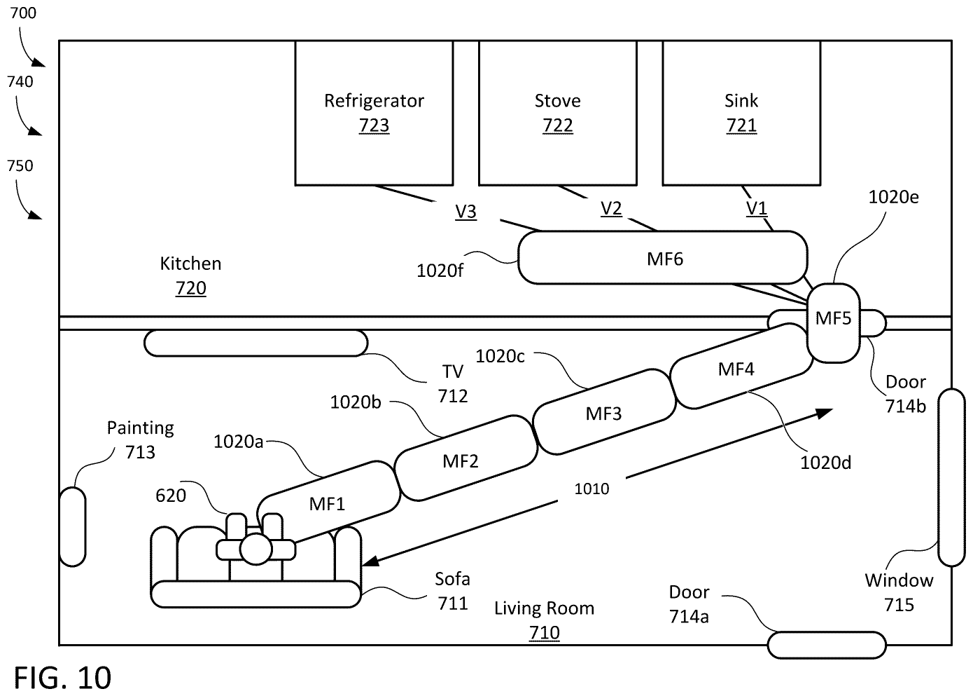

FIG. 10 illustrates an example of a PUM 620 in a SEE 700, with an initial start point, for example sitting on a sofa 711, undertaking a set of movements which are characterized by a set of movement frameworks 1020a-d (generally or collectively, movement frameworks 1020), towards the interconnecting door 714b. The movement framework MF5 1020e represents the PUM 620 traversing the door 714b, and the movement framework MF6 1020f represents the PUM 620 having a choice as to which focal point that the PUM 620 will move towards. These potential movements may be represented by a set of vectors, V1, V2 and V3 which are aligned with the behavior paths (820b-d) for the respective focal points 750 of the sink 721, stove 722 and refrigerator 723. In some embodiments, movement framework MF6 102f may be classified as a decision point or mobility language connector. In the same manner, movement framework MF5 1020e may be classified as a reductionist movement framework due to the smaller number of movement options once commenced.

In some embodiments, one or more AI/ML systems may be used to predict the likely behaviors of a PUM in a SEE, and may use a PPE to represent the anticipated movements of the PUM in response to these behaviors. In some embodiments, this combination of PPE, AI/ML systems and SEE, where the SEE includes a set of mapped focus or attention points and the PUM behaviors are represented, at least in part, by the one or more sensors, devices or systems present in the SEE generated data sets or patterns, can provide an overall monitoring framework for a PUM, which when deployed in one or more digital twins can enable and support accurate and timely health, care safety or wellness supporting operations.

One aspect of this approach may be the development of the capability to ascertain the timing or degree of physical or mental changes and variations that a PUM may be undergoing. One potential aspect of any effective health, care, safety or wellness monitoring systems is the reliable capability to predict when a health, care, safety or wellness event that is detrimental to a PUM may occur. These data can then be used to mitigate or avoid such an event. For example, falls in the elderly that result in hip fractures often leading to the early demise of the PUM. An effective and efficient monitoring system employing a PPE, AI/ML systems, or digital twins can provide predictions of such an event, and as such preemptive actions may be taken to remove or reduce the risk of this occurring. The present disclosure, when applied with the HCP of the PUM, may therefore provide for the prophylaxis or treatment of the identified conditions, diagnosis or suggestion of additional or developing medical conditions, and the provision of identified (e.g., in the HCP) of specific pharmaceutical compounds or therapies for the prophylaxis or treatment thereof.

This improvement in the care of the PUM can particularly be observed in the case where cognitive decline of a PUM is present, such that the PUM is less able to make effective decisions regarding personal health, care, wellness or safety. For example, if the PUM being monitored is observed to have increased confusion, indecision or other impairments in regard of intentions or attentions, for example, through monitoring of behaviors of the PUM in movements and actions regarding such focus and attention points mapped within a SEE, then the care hub or care processing systems may generate messages, alerts or communications to the PUM or other stakeholders, including carers as to this deterioration. In many circumstances, such deterioration is gradual and incremental, with neither the PUM nor responsible stakeholders noticing or quantifying the reduction in capabilities of the PUM. This gradual deterioration of the capabilities of the PUM can lead to the first realization being caused by a health, care, safety or wellness event, such as a fall, an overflowing bath, leaving domicile in an uncontrolled manner (e.g., wandering), having a fraud perpetrated upon the PUM and the like.

The use of the PPE, AI/ML systems, digital twins or game theory enabled monitoring systems providing predictive data sets as to the likelihood of this trajectory of reduced capability can inform the PUM, stakeholders of the PUM, care hubs and care processing systems and any other responsible entities of this deterioration. This trajectory determination enables one or more preventive strategies to be employed for the benefit of the health, care, wellness or safety of the PUM.

For example, one preventive or remedial approach may include the use of one or more sensors, devices or systems to generate, at least in part, one or more messages or communications, which can include visual, audio or haptic data in any arrangement, so as to guide the PUM's attention to a specific situation. This guidance can include reminders for taking medicines, eating, consuming entertainment, attending to social interactions, exercising or other activities or events or providing, for example though haptic communications that the PUM focus attention on a specific part of the SEE, for example a door, window, kitchen, bathroom and the like. This guidance can include providing prompts to the PUM where the behaviors of the PUM have deviated sufficiently from an established and monitored routine, for example when the observed behaviors, including patterns, mobility modules, mobility frameworks, in which case, for example, the PUM may be alerted to such a deviation in a manner that is not deemed invasive by the PUM. This manner may include, in some embodiments, the use of one or more generative AI system deployments, including for example a generative AI that is configured for the specific PUM.

Such attention guidance can be non-deterministic, such as encouraging the PUM to move outside the domicile environment, such as to a garden, to, for example, increase vitamin D intake and potentially incur mental stimulation from that environment. In this example, the attention is not focused on a single event, activity or location, rather there is a cone or sphere of attention that is directed into a general area, from which the PUM may garner multiple benefits.

In some embodiments, the evaluation of the intention or attention of the PUM through, for example, gaze or eye monitoring or the one or more data sets, patterns or behaviors determined, at least in part, by the sensors, devices or systems present in the SEE may include one or more risk variables, which can be calculated, for example, through the use of a PPE, AI/ML systems, game theory modules or digital twins in any arrangement. Risk assessment can include probabilities derived from PUM movements, their gaze or head alignment or focus and attention points, as calculated through use of PPE, AI/ML systems, game theory modules and digital twins in any arrangement.

In some embodiments, one or more systems may operate to compare the observed data sets or patterns generated by the one or more sensors, devices or systems and compare these data sets with the data sets generated by one or more AI/ML systems that are representing such one or more sensors, devices or systems. In this example, a PPE may provide physical characteristics of the environment or the PUM therein, such that the AI/ML generated data sets or patterns may be aligned in accordance with the physics present at the time, for example, using a PPE. For example, if the AI/ML systems generates a data set that includes attributes that exceed the local laws of physics, then this data set can be constrained by the PPE to reduce such a variable to an appropriate value. In this manner the PPE may form part of a RAG.

In some embodiments, sets of characteristics of other stakeholders, such as carers, family, neighbors and the like may be used to configure a PPE, such that the PPE can represent these other stakeholders. For example, there may be multiple instances of PPE operating in a SEE, where for example there is a PPE operating that represents the PUM and a further PPE representing another stakeholder, for example, a carer. In this example, a care hub or care processing system may, using such multiple PPE, calculate the interactions of the persons represented by such PPEs. For example, the care hub or care processing system may alert a carer that the PUM cannot reach an object, whereas the carer can.

### USE OF GAME THEORY

One aspect of the SEE may be the monitoring of the state of the PUM and the environment, where for example, a PPE may form part of that monitoring arrangement. In some embodiments, there can be games theory games that have strategies or operations intended to identify, at least in part, changes in the state of a PUM in a SEE. For example, if a PUM is undertaking a daily routine, which in some embodiments may be represented by one or pattern frameworks, which can be populated by one or more data sets for the one or more sensors, devices or systems included in such SEE, where for example PUM or other stakeholder behaviors can be expressed as tokens, including those behaviors that are in a quiescent state, or which can be represented by bevokens, one or more games may be operating to determine the potential that a PUM behavior or state is changing. This determination can include games that operate over extended periods, for example months, whereby although the data sets represented by the tokens are within the thresholds of those tokens, the deviations within those data sets can be represented by variations in the strategies employed by the one or more sensors acting as players in those games. For example, if a sensor data set includes increasing or decreasing values toward a threshold or exhibits increased volatility in the data sets, this trend can impact the strategy of the sensors in, for example the game of accuracy, timeliness or resource cost that the sensor may be undertaking.

One aspect of the deployment of one or more game theory modules and the games thereof, may be the configuration of those games to represent the choices of a PUM. This configuration can include the possible alternatives available to a PUM, including those constraints imposed by the physical constraints of one or more health, care, safety or wellness conditions, which may form part of a HCP for the PUM.

One or more PPE or digital twins may, in some embodiments, be configured to operate with one or more game theory modules to represent the specific physics of these options, where, for example, one or more games may unfold as part of a predictive analysis of the PUM behaviors.

In some embodiments, such predictions can be made available to a PUM or to other stakeholders representing alternatives that may be available to the PUM. This provision of the predictions can include the use of one or more tokens as representations of such alternatives. For example, if the PUM has a behavior which becomes less and less possible due to, for example, reduced mobility, the PPE in combination with one or more game theory modules or in collaboration with one or more care hubs or care processing systems, may present to the PUM alternative behaviors to satisfy the intentions of the PUM. For example, if a PUM is determined to have the intention to watch television, but has suffered a hip fracture, and can no longer walk from a current location to where the remote control is located, the systems may generate a reminder in the environment that the PUM should use audio commands to activate the television, call for assistance from a caretaker to retrieve the remote control for the PUM, or activate the television on behalf of the PUM without receiving further commands or instructions.

One aspect of this selection of or presentation to alternatives to a PUM may be the state of the PUM's well-being at that time, in that if the PUM has a state of frustration, discomfort, angst or other behavior, which is affecting to a greater or lesser degree the well-being of the PUM, which in some embodiments may be determined, at least in part by data sets or patterns generated by the one or more sensors, devices or systems present in a SEE, a PPE, potentially in collaboration with one or more care hub or care processing system, may constrain, reduce, emphasize, highlight or recommend one or more particular game outcomes or strategies in light of such circumstances.

In some embodiments, one or more game theory games may be deployed to ascertain, at least in part, the attention or intention of a PUM. For example, a set of games, representing PUM behaviors where the PUM is located at a location within the SEE and a set of potential behaviors are represented as a set of games, where each of the potential behaviors can have a destination corresponding to a particular behavior. In this manner, there may be a set of games which represent the potential behaviors of a PUM from a location within a SEE. This can include selection of behaviors exhibited previously by the PUM, including those that are undertaken at specific times of the day.

In some embodiments, a PPE can use AI/ML-based game-like strategies such as adversarial neural networks to generate, validate and improve the PPE simulations. Adversarial Neural Networks (ANNs) incorporate the concept of adversaries, inspired by the game-theoretic idea of minimizing losses through competition. An example of ANNs may be Generative Adversarial Networks (GANs) that use an adversarial process for improving generated data.

In a typical GAN, there are two neural networks: the generator and the discriminator. The generator tries to create data (e.g., images) that appear to be real, while the discriminator attempts to distinguish between genuine and generated data. The generator and the discriminator may train simultaneously in a zero-sum game where one's gain is another's loss. This adversarial process leads to both networks improving over time through backpropagation of gradients, ultimately resulting in high-quality synthetic data generation by the generator that can be nearly indistinguishable from real data.

To use Adversarial Neural Networks (ANNs) for validating a neural network-based physics simulator, as part of a PPE, an approach similar to Generative Adversarial Networks (GANs) can be used, by having an AI/ML model that serves as a physics simulator capable of generating synthetic data from sensors, devices or systems input, based on physical laws and principles, as well as the PUM's movement patterns, mobility modules, mobility frameworks, behaviors or environment characteristics, modeled by neural networks. This model would be the generator. Another AI/ML model designed to distinguish between the simulator's output and real-world observations of similar physical systems can be used as the discriminator.

For example, the generator can learn how to create data that are indistinguishable from actual data, gathered from one or more PUMs and one or more SEEs, in terms of physical consistency, statistical properties, or realism. The discriminator can accurately judge whether the generated synthetic data are plausible within a given set of physical constraints (for example, conservation laws, known relationships between variables and the like).

Both AI/ML models can be trained in an adversarial manner by continuously updating each model's parameters based on the performance of the other model using, for example, back propagation. The generator improves the ability to create realistic simulations by learning from the discriminator's feedback about how well the generator approximates reality, while the discriminator learns to better identify subtle inconsistencies or anomalies in both synthetic and real data samples.

The validity of the physics simulator may be assessed by evaluating whether the datasets generated by the physics simulator are capable of fooling the trained discriminator into classifying the generated datasets as ‘real’ or from actual observations. If the discriminator cannot consistently differentiate between real and synthetic data, this lack of consistency indicates that the physics simulator has learned to replicate physical phenomena accurately. By iterating through this adversarial training process, both models can be refined to reach a point where the generator produces high-fidelity simulations that are virtually indistinguishable from real-world data as judged by the discriminator.

In some embodiments, a PPE using representations of the movements of a PUM, for example in the form of pattens, mobility modules or mobility frameworks, may in collaboration with one or more AI/ML system predict one or more behaviors of the PUM. For example, if a PUM stands from a sitting position and walks towards a kitchen, the PPE and AI/ML combination may predict based, at least in part, on the time of day, that the PUM is about to undertake a specific behavior, for example making a coffee or another behavior that may occur in this time period. This prediction can, for example, involve the use of one or more digital twins of the SEE or the PUM, represented by a PPE, where the range of potential behaviors has a reduced scope based, at least in part, on the location of the PUM, the time of day or the previous PUM behaviors. One aspect of this approach may be the determination of the sequence of movements, for example represented by mobility frameworks that include a behavior, such that the PUM may vary the initial set of mobility frameworks that start a behavior in light of one or more physical attributes, such as knee, back, shoulder or other body pain or discomfort. As the PPE can represent the potential behaviors, including representing new behaviors that, for example, include patterns, mobility modules or mobility frameworks or sets thereof in sequences that have not been observed previously, the PPE can, for example, generate one or more data sets that can be communicated to, for example, one or more sensors, devices or systems, including for example care hubs or care processing systems, one or more stakeholders, including for example the PUM or one more other systems or modules, such as for example one or more AI/ML systems, game theory modules or the like.

The use of a PPE to establish a set of predictions for the behavior of a PUM may be used to establish, for example, trends of the behaviors of the PUM over various time periods. For example, the identification of longer-term trends, when over an extended period a PUM has a deterioration of one or more aspects of physical or mental well-being, for example as the effects of arthritis, injuries, deterioration of eyesight, mental acuity deterioration and the like, which involves multiple data sets over the extended time periods. The PPE can be used as the basis for comparison between and among the data sets because each of the data sets generated by the one or more sensors, devices or systems of or present in the SEE to provide data to the one or more monitoring systems, including care hubs or care processing, can be processed by the PPE. These data sets may be compared to the PPE which can represent, for example, the mobility capabilities of the PUM from previous time periods, enabling the evaluation of the current data sets to identify any divergence from one time to another. An instance of the PPE may be generated based on these original time period data sets, for example when the PPE is initialized, and can consequently provide a frame of reference for any future data sets. Further PPE may be instantiated at various times, for example when a health event has occurred, for example a knee replacement or the like, or on a periodic basis, for example every six months. These PPE can then be evaluated to further identify any trends in the mobility, mental acuity, decision making or other physical or mental aspects of the PUM. This longitudinal analysis can be particularly informative in regard of gradual deterioration of mental acuity or similar physical deterioration, where these trends may be presented to one or more stakeholders, including the PUM. For example, the PPE may, based at least in part on these trends, communicate to one or more stakeholders, including the PUM, that the current observed or predicted trend is likely to result in a detrimental health, care, safety or well-being incident, for example a fall. For example, such trends may inform a carer or relative that the PUM should cease driving a vehicle in light of the PUM's reduced mental acuity. In such situations, one or more preventive, mitigating or remedial actions may be undertaken by the one or more stakeholders.

In some embodiments, a PPE may provide one or more messages or other communications, including for example, as tokens, to one or more sensors, devices or systems present in a SEE. For example, a PPE which has identified or predicted a PUM initiating one or more patterns, mobility modules, mobility frameworks, behaviors or other activity, may communicate a set of data, including calibration, configuration or other data arrangements, to one or more sensors, devices or systems present in a SEE. For example, if a PUM has initiated a set of mobility frameworks and these mobility frameworks are identified by a PPE as those mobility frameworks forming the initial sequence of one or more behaviors, the PPE may directly or indirectly, for example though one or more care hub or care processing system, communicate a set of configuration data to those sensors that are in the path of or can evidence the behaviors of the PUM. In this manner, the behavior of the PUM can be established with the relevant sets of sensors that are most suited to that recognition. This approach can include evaluation of the one or more sensors, devices or systems data sets, in that the combination of data sets, for example those predicted by the PPE, may vary from the actual data sets, for example, if a sensor is, at least in part, obscured by an insect, dust or other impediment to that sensor, device or system capabilities.

Accordingly, the configuration data may be used to activate and deactivate various individual sensors, but also to configure already active sensors to gather or report data in different ways. For example, a camera configuration may be used to move (via one or more motors) where the camera is focused in the SEE, a change depth of focus in the SEE, activate various light sources or audio sensors associated with the camera, change a compression algorithm applied to images or video collected by the camera, increase or decrease a framerate at which the camera collects or reports video or images, adjust whether post-processing effects or determinations are made on the collected videos or images before reporting the videos or images (e.g., bounding boxes, wireframes, skeletal models, etc.), or the like and combinations thereof.

The PPE may directly or indirectly provide data sets representing the predicted or actual movements of a PUM, for example, in the form of mobility frameworks, which can be represented by one or more tokens. These data sets (including tokens) may then be employed for the calibration, configuration, correlation, alignment or other instructions to the one or more sensors, devices or systems present in the SEE. For example, a PPE initiated communication may include data that instructs one or more sensors, devices or systems to detect, identify or respond to one or more data sets generated by such one or more sensors, devices or systems, for example by communicating such data to one or more care hubs or care processing systems, where, for example, such systems may invoke one or more responses, such as alerting a carer or other stakeholder, communicating with the PUM, through, for example, audio, visual or haptic means. In some embodiments, such PPE communications can include feedback loops where the PPE can initiate or receive feedback to or from one or more sensors, devices or systems to, for example, validate, augment or verify those data sets.

In some embodiments, a PPE may through representation of the mobility of the PUM collaborate with the PUM in their undertaking of such mobility. For example, a PPE may monitor the PUM movements, and through representation of those movements determine a set of movements that can be beneficial to a PUM. For example, the PPE may invoke one or more digital twins or one or more AI modules to evaluate the manner in which a PUM moves from one position to another, for example from sitting to standing. In this manner, the PPE may determine one or more mobility frameworks involving a particular set of skeletal or muscular movements that can be beneficial to a PUM. For example, if the PUM has arthritis or another physical constraint, the PPE may propose a different initial mobility framework that puts less stress on the PUM to achieve the same intent. These proposals can then be communicated to the PUM or other stakeholders through one or more devices or systems, including worn, implanted or carried devices, such as smart phones and the like or through those devices or systems embedded in the environment, such as a smart TV and the like. These communications can involve audio, video, haptic or textual communications in any arrangement.

This approach may include the PPE providing the PUM or other stakeholders with representations of movements, for example, in the form of mobility frameworks, that can be communicated to a PUM, for example through visual, audio or haptic methods in any arrangement. For example, such an approach may be deployed to enable or encourage a PUM to undertake specific sets of movements or exercises for the overall health, care, safety or wellness benefit of the PUM.

In some embodiments, sets of measurements generated by the one or more sensors, devices or systems of a SEE monitoring a PUM can be aligned with the PPE of that PUM, where such measurements are represented by, for example, mobility frameworks. These mobility frameworks may be configured as tokens, which can be distributed to one or more sensor, devices or systems or other stakeholders, such that the data sets including such tokens may calibrate, configure, inform, align or in other manners provide data sets to the recipient of the tokens. These tokens may be encoded such that only the recipient specified by the dispatching system, for example the PPE, may access the data within the token. In various embodiments, the token is a formatted data object configured for ingestion by or according to output parameters of an AI/ML model or layer thereof.

In some embodiments, multiple PPE, representing multiple PUM may generate mobility frameworks that are representations, at least in part, of each of the PUM behaviors. These behaviors may form an ontology, taxonomy or other form of organization or arrangement, such that the individual mobility frameworks including each such behavior may be compared and evaluated across multiple PUM. These evaluations may inform one or more stakeholders, including a PUM or one or more sensors, devices or systems as to the likely behaviors of one or more PUM based, at least in part, on such evaluations. This data set, generated by the one or more PPE, can be employed by one or more AI/ML system as training data to generate, at least in part a composite model representing the potential behaviors based on one or more sets of mobility frameworks. Such behaviors can be employed with one or more game theory systems to establish the steps in one or more games, represented by the, for example, mobility frameworks exhibited by the one or more PUM as represented by a PPE. For example, such games may include time of day, location, environment and other contextual features, which can impact the strategies of a player in that game, such as one or more sensors, devices or systems, a PUM or other stakeholders. The outcome of such games can include communications to the one or more sensors, devices or systems, stakeholders, including the PUM in any arrangement.

In some embodiments, a PPE can represent a set of common injuries that typically impact a PUM, for example, knee, hip, wrist or other joint issues, arthritis, vision impairment, hearing impairment and the like. This representation can include, for example, mobility frameworks that represents a broken hip that has been caused by a fall, or a broken wrist caused by arresting a fall or the like. Such mobility frameworks, representing these injuries can be used by a PPE to evaluate the current state of a PUM to determine if that injury has occurred or issue an appropriate alert or to predict such an injury occurring if the preceding mobility frameworks are indicative of such injury. For example, after detecting a suspected fall event using haptic, acceleration, audio or other sensors, the PPE may continue to monitor the behaviors and movement frameworks identified via visual sensors, accelerometers and the like in the environment to compare the PUM's actions to the representations of various injuries. Using these comparisons, the PPE can update a determination of “fall with potential injury” to “fall with no apparent injury”, “fall with potential injury to wrist”, “fall with potential injury to ribs”, etc.

In some embodiments a PPE may be employed with one or more AI/ML systems in the form of a RAG, where the PPE can provide a set of constraints to an AI/ML systems, including for example LLM's, to restrict the output of the AI/ML systems to those possibilities that are physically possible in the context of the environment or the physical state of the PUM. For example, an AI/ML system may, without constraint, identify a potential behavior of the PUM as performing calisthenics including performing the splits based on a body and limb position of the PUM, which the PPE constrains as not physically possible for the PUM, to thereby cause the AI/ML system to recognize an alternative behavior of the PUM (e.g., a fall, a sensor error, or the like).

### FLOWCHARTS

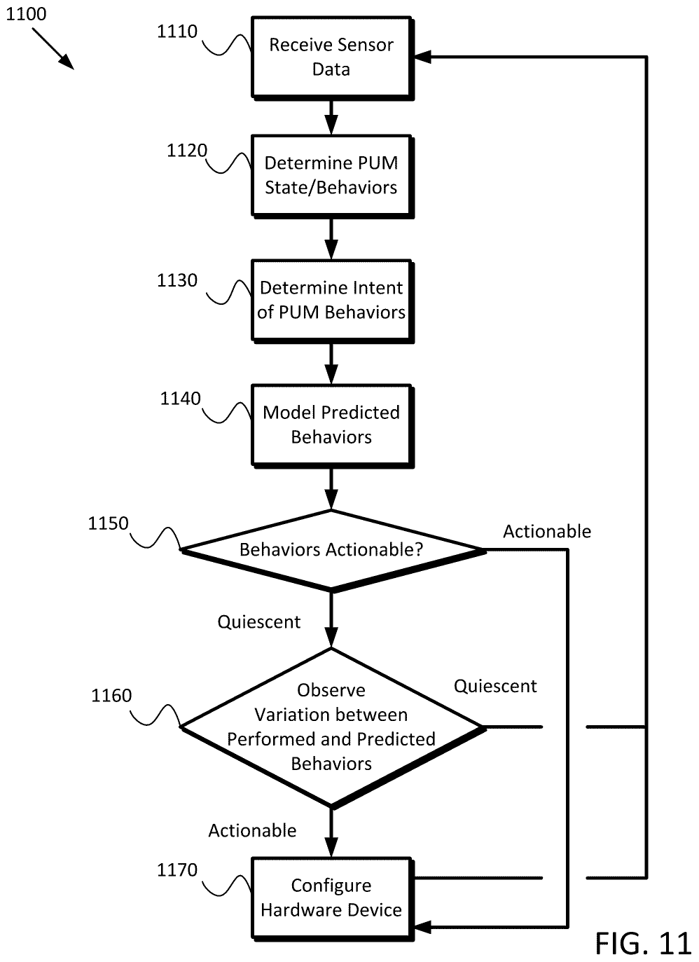

FIG. 11 is a flowchart of an example method 1100 for employing a PPE for improving the care of a PUM in a SEE, according to embodiments of the present disclosure. Method 1100 begins at block 1110, where the system receives sensor data from one or more environmental sensors disposed in a SEE. The sensor data may be received in various formats, including encrypted or unencrypted and raw or processed. In various embodiments, the sensor data are processed and layered in encryption as tokens, which provide a wrapper that indicates a processed determination of a behavior observed in the SEE in a first layer of encryption (which may include unencrypted) and raw data, sub-tokens, or links to raw data in a second layer of encryption. In various embodiments, a token may combine the sensor data from two or more sensors or tokens produced by two or more sensors, which provides for modularity and multi-sensor data fusion from a chain of determinations.

For example, the system may receive a first token that indicates, in a format configured for ingestion by an AI/ML model, that a fall event has occurred in the SEE in a wrapper encrypted according to a first encryption key. In the payload of the first token, raw data from a first environmental sensor (encrypted according to a second encryption key) may be present from an audio sensor, as can a second token processed by a PPE or other system that indicates that a PUM engaged in a rapid lying down behavior (which in turn can include the raw sensor data from various environmental sensors that the rapid lying down behavior was identified based on). The conclusion indicated the first token that a fall event occurred, rather than the PUM engaging in calisthenics, controlling decent to the indicated pose (in the second token) may be based on the audio data exceeding a noise threshold and the behavior of the PUM. Accordingly, the tokenized data provide a chain of evidence for the various determinations made in the system, as well as layered privacy to different aspects of the data used to make those determinations.

In various embodiments, the sensor data may be received at different rates and at different granularities or fidelities at various times. For example, when the system identifies that no one is present in a given section of the SEE, the sensor data may be received at a reduced frequency from environmental sensors in that section of the SEE compared to when someone is identified as being present in that section (e.g., a bedroom when a PUM is sleeping at night or napping during the day). For example, when the system identifies that an event has occurred, such as when an audio sensor or motion sensor indicates that activity is occurring in a section of the SEE, one or more environmental sensors may be activated or signaled to increase a data reporting rate.

Time of day may also be used to affect the signaling rate or data fidelity or granularity of the data reported by the environmental sensors. For example, the environmental sensors in a bedroom may have a first reporting configuration when no one is present is the bedroom, a second reporting configuration around an expected bedtime and wake time, and a third reporting configuration between the expected bedtime and wake time when the PUM is expected to be asleep, and may require lower fidelity or frequency of monitoring compared to when falling asleep or waking. Accordingly, the system can configure an amount of data to process based on the needs of the PUM and other stakeholders for the care of the PUM while conserving computing resources and transmission bandwidth.

At block 1120, the system determines, using the sensor data received per block 1110, a state of the PUM and behaviors of the PUM. Because method 1100 describes an ongoing and iterative process in which the sensor data may be received at different rates with various amounts of processing applied thereto, the sensor data may include data received across various time windows and various previously determined states or behaviors that are used to inform a current determination of state or behavior of the PUM. The PPE may also model the physical potential of the PUM to differentiate between various behaviors of the PUM.

For example, sensor data received at a first time may indicate that a PUM is in a standing state, while sensor data received at a second time may indicate that the PUM is on the ground in a lying down state. Using the data from the two times maybe sufficient to indicate a lying down behavior or a falling behavior, which an AI/ML model may validly choose between for a PUM based on past behavior or conditions indicated in an HCP. For example, the model may indicate that a first PUM performed a lying down behavior (rather than a falling behavior) when the first PUM is noted as frequently performing tasks while lying down (e.g., regularly stretching, cleaning the floor, playing games on the floor, etc.), but may indicate that a second PUM performed a falling behavior (rather than a lying down behavior) when the second PUM is noted as a fall risk in an associated HCP. The PPE may also distinguish between potential behaviors based on the laws of physics and the speed and range of motion associated with the PUM modeled by the PPE such that a spry first PUM is more likely to be identified as performing a rapid (but controlled) change from the standing state to the lying down state as a “lying down” behavior than a generally less mobile second PUM, who would be more likely to be identified by the PPE as performing a “falling” behavior to achieve the same change in state in the same time period as the first PUM.

Behaviors may also be updated or revised based on subsequently received sensor data. For example, as additional states are received for the PUM, the system may refine or expand behaviors or behavior frameworks. For example, when a rapid succession of “standing” and “lying down” states are received, the system may identify the series of behaviors as “exercising” rather than as multiple determinations of “standing up” and “lying down” behaviors. For example, after initially determining that the PUM engaged in a “lying down” behavior rather than a “falling” behavior, additional data of the PUM calling out for help may be used to revise the determination of the behavior to a “falling” behavior.

At block 1130, the system determines the intent of the PUM based on the state and the behaviors of the PUM determined per block 1120. Because method 1100 describes an ongoing and iterative process in which the state and behavior determinations be made at different rates with various revisions applied thereto, the states and behaviors may include determinations made across various time windows. The PPE may also model the physical potential of the PUM to differentiate between various intents of the PUM.

In various embodiments, a position of some or all of the body of the PUM in the SEE (e.g., a state) may indicate a focal point for an intended set of behaviors. For example, a PUM who is determined to be located on a sofa and facing a TV may be determined to have an intent of watching a TV show.

In various embodiments, one or more actions of the PUM in the SEE (e.g., a behavior) may indicate the intent of the PUM. For example, a PUM who is walking from a sofa towards a door into a kitchen may be determined to have an intent to interact with an element in the kitchen, such as getting a drink from a faucet or refrigerator, getting food from a refrigerator or pantry, washing hands in a sink, cleaning the kitchen, etc.

In various embodiments, one or more game theory games are used to identify an intent of the PUM using predicted actions for a digital twin of the PUM to achieve one or more goals or positive outcomes in a game theory game. For example, the system may use a game theory game to determine which one movement of a plurality of potential movements follows a particular movement in the series of predicted movements that can form at least one behavior based on a reward framework for matching behaviors to the intent. For example, to retrieve an item, the PUM may be modeled as scooting while in a seated position to a new location on a sofa, standing up and walking to a new location, or calling for an assistant or service animal to retrieve the item-all of which could achieve the intent of item retrieval, but have different costs and rewards according to a game theory model, which may model the highest payout to identify which strategy the PUM will employ.

In various embodiments, one or more previously determined intents may be used to determine a current intent of the PUM. For example, if the PUM is determined at a first time to have an intent of watching TV, and then rapidly changes focal points in the environment and rises from a sitting state to a standing state, a second intent for “retrieving a remote control” may be identified.

In various embodiments, because accidents are events that occur without intent, the determined intent may persist through a series of events or be made independently of the events occurring after the determination. For example, if the PUM falls (e.g., rapidly transitioning to a lying down state while not intending to do so), the system may note that the PUM fell while intending to walk from the sofa to the kitchen. For example, if the intent of the PUM is to be to retrieve a television remote from a shelf in order to watch TV, but an environmental sensor indicates that a book fell or was removed from the shelf, the intent of the PUM may remain “retrieve remote control” or “watch TV” rather than being updated to “read book”.

In various embodiments, the intent may be used to inform further determinations of behaviors. For example, a PUM who is determined to have an intent of exercising may be less likely to be identified as “falling” compared to “lying down” when performing the same actions.

At block 1140, the system models predicted behaviors of the PUM based on the sensor data, state, behaviors, and intent for the PUM.

At block 1150, the system determines whether any of the predicted or observed behaviors are actionable. When the behaviors are identified as quiescent, method 1100 proceeds to block 1160 for further analysis, and when the behaviors are identified as actionable, method 1100 proceeds to block 1170 to engage a hardware device associated with follow on actions for the behavior to manage or affect the behavior of the PUM or other stakeholder associated with care of the PUM.

Various behaviors may be determined to be actionable without further input or analysis. For example, an observed behavior of the PUM falling from a ladder or a predicted behavior of an epileptic episode may result in a response being generated. Further analysis may continue according to method 1100, but the rapid generation of a response to the observe actionable behavior improve the care of the PUM.

Various otherwise innocuous behaviors may be determined to be actionable according to one or more HCP triggers defined in an HCP for the PUM despite the displayed behaviors ordinarily being quiescent. For example, a behavior accompanied by heart rate that falls outside of an acceptable window of values may be indicative of a heart failure or tachycardia, and should result in an action by the system to alert a stakeholder regardless of other behaviors identified. For example, behaviors of the PUM sitting down in a controlled manner may not ordinarily be indicative of an actionable behavior, but when accompanied by an arrhythmia may be indicative of a medical condition for which a response is needed. For example, behaviors of the PUM walking from a sofa to a kitchen may not ordinarily be indicative of an actionable behavior, but when accompanied by tachycardia may be indicative of the PUM experiencing a fright (e.g., due to a condition not identified by the environmental sensors) and for which a response is needed. Accordingly, when an actionable threshold is reached.

In various embodiments, an HCP trigger corresponds to movement of a joint of the PUM identified in the HCP with pain or reduced efficacy of movement for the PUM, such that behaviors that include movement of the identified joint are identified as actionable behaviors so that a response, message or alert may be generated to include a suggested alternative behavior for achieving the intent with reduced pain or with improved efficacy of movement for the PUM.

In some embodiments, when modeling the series of predicted behaviors per block 1140 generates a path through the SEE), identifying an actionable behavior includes evaluating the path for the presence of an HCP trigger identified in the first HCP (e.g., a tripping hazard based on the PUM's modeled gait) so that a response, message or alert may be generated to include a suggested alternative path for achieving the intent or removal of the HCP trigger from the path in response to detecting the HCP trigger.

In various embodiments, the HCP may identify various HCP triggers for actionable behaviors or environmental conditions, which may include one or more of; a tripping or fall hazard; a repeated movement through the SEE towards and away from a focal point identified with the intent; a destination point of the series of predicted behaviors not associated with the focal point identified with the intent; a false-start motion in the series of performed behaviors absent from the series of predicted behaviors; and a pain-inducing motion in the series of performed behaviors absent from the series of predicted behaviors.

Further analysis may continue according to method 1100 to identify or update any other actionable behaviors, but the rapid generation of a response to the observe actionable behavior improve the care of the PUM. Accordingly, more immediate responses may be generated, to thereby improve the care of the PUM when various actionable criteria are met.

In various embodiments, the quiescent state represents the behaviors and movement frameworks that do not cause the system to generate an alert or other follow-up action. For example, a behavior of a PUM sitting down to watch TV at three in the afternoon may be considered a quiescent state for the PUM whereas sitting down to watch TV at three in the morning may not be considered a quiescent state for the PUM based on the observed wakefulness patterns for the PUM (e.g., indicative of a disrupted sleep pattern). For example, a behavior of a PUM vigorously using a stationary bicycle at nine in the morning may be considered a quiescent state for the PUM whereas the PUM lackadaisically using a stationary bicycle at nine in the morning may not be considered a quiescent state for the PUM based on the observed exercise patterns for the PUM (e.g., indicative of poor health preventing vigorous exercise). For example, a behavior of a PUM more noisily or forceful impacting a surface than a threshold deviation from previous noises or forces of impact with that surface may be indicative of a fall or arrested fall, which the system may treat as a non-quiescent behavior for which follow-up actions are initiated.

At block 1160, the system determines whether a level of variance between predicted behaviors and observed behaviors are actionable. In addition or alternatively to triggering criteria resulting in a response being generated for actionable behaviors, the system monitors a variance to identify when otherwise innocuous behavior may be actionable based on the behavior being unexpected or indicative of a mental or psychological condition not otherwise observable physically in the PUM. For example, if the PUM is observed as moving back and forth in the SEE, such behavior may be innocuous or quiescent (e.g., indicative of the PUM exercising, searching for an object, chasing a pet, etc.) when the actions match the intent and predicted actions for achieving that intent, but may be actionable when the actions do not match an intent (e.g., indictive of a dementia event, involuntary movement of limbs).

In various embodiments, the PPE may be used to filter the actions of the PUM so that involuntary movements of the PUM due to tremor are discounted or weighted less heavily (including ignored) when assessing the level of variance between predicted behaviors and observed behaviors. For example, a predicted be behavior of a PUM to take a drink may be affected by the level of jitter in the hands of that PUM, and may take longer (or result in false starts, dropped cups, or the like) when the PUM is noted to experience this level of jitter. Accordingly, the PPE may be configured to ignore movement below a gross motor threshold when identifying whether the variation between a series of performed behaviors and a series of predicted behaviors exceeds the actionable threshold.

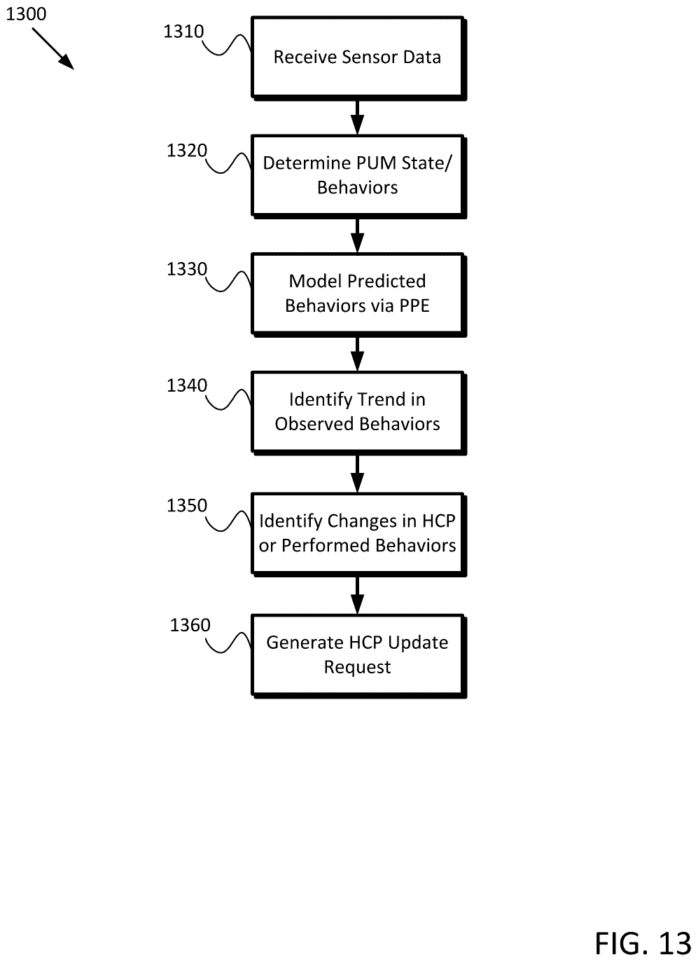

The level of jitter that a PUM is expected to display, and is thereby modeled as displaying by the PPE, may be identified in an HCP or learned over time. The PPE may longitudinally track this level of jitter to identify when a level of jitter changes over time and should become actionable. Accordingly, the PPE may be configured to monitor a severity or frequency of jitter as part of monitoring care of the PUM, which may include operations as set forth in method 1300 discussed with respect to FIG. 13.

At block 1170, the system engages one or more hardware devices associated with the SEE or PUM based on the actionable determination in one or both of block 1150 and block 1160 to update a configuration of the hardware device. The hardware device may be identified from a plurality of configurable devices in the SEE that the system is in communication with, and the identification may be based on one or more of the intent, state, performed behavior, or deviation of modeled behavior from performed behavior. In various embodiments, (re)configuration of the hardware device is selected to affect a behavior of the PUM or other stakeholder for care of the PUM or engage in treatment of an observed medical condition according to the HCP by reconfiguring or performing another action via that hardware device. In various embodiments, configuring the hardware device may include calibrating the device or the data reported thereby to according to external calibration rubric, causing the hardware device to self-calibrate, causing the hardware device to return to a preset configuration (e.g., factory settings), or causing the hardware device to update settings according to a calibration file provided by the system.

In some embodiments, the system generates and transmits message via a computing device associated with the SEE. In various embodiments, the message may be transmitted to one or more stakeholders in the care of the PUM, which may include the PUM, a family member of the PUM, a friend of the PUM, a neighbor of the PUM, a designated health, wellness, or safety contact of the PUM (e.g., according to an HCP), a doctor, a nurse, a healthcare assistant or other medical professional or provider, an emergency services provider, a care animal, a living facility attendant, or the like for any person or entity acting as a carer to the PUM. The message may be transmitted to a computer, cell phone, landline or software phone, pager, intercom, tablet, animal collar, or other device designated as having a relationship or otherwise being associated with the relevant stakeholder.

In various embodiments, the individual stakeholders to whom the message is transmitted may be based on the nature of the actionable behavior, whether the actionable behavior was predicted or observed, the intent determined for the PUM, triggering conditions in the HCP, and combinations thereof. For example, a physical therapist (as a stakeholder) may be altered as a confirmation when the PUM is identified as engaging in exercises when the HCP for the PUM is set up to include an HCP trigger to identify when the PUM engages in exercise. For example, when a PUM is predicted to experience an epileptic episode (but has not yet been identified as seizing), a first response may be transmitted to the PUM to alert the PUM to the predicted behavior, and a second response may be transmitted to issue a command to a service animal to come to the PUM's aid. Continuing the example, a third response may be transmitted to a home care assistant of the PUM if the PUM begins to seize, but may be omitted if the prediction is a false positive and the PUM does not seize within a predicted time window.

In various embodiments, the message may include text messages, video messages, audio messages, haptic messages and combinations thereof that are transmitted via various open or proprietary standards. In some embodiments, the message can include one or more tokens.

In some embodiments, in addition or alternatively to transmitting a message, the system may engage various other hardware devices located in or having a relationship with the SEE. For example, the system may engage an action in the hardware device to: adjust a temperature of a thermostat for an HVAC unit, space heater, radiator, or the like associated with the SEE; adjust a speed of a fan disposed in the SEE; adjust a brightness of a light in the SEE (e.g., turn on, turn off, dim, brighten, adjust wavelength); turn off or on an appliance (e.g., oven, stove, sink, microwave, toaster, washing machine, clothes dryer, dishwasher, water heater, television, radio, etc.) in the SEE; connect or disconnect water or gas from a source to one or more fixtures (e.g., sink, toilet, shower, dishwasher, oven, stove, refrigerator, bubbler, water heater, etc.) in the SEE; adjust a volume, channel, brightness, contrast, or content item provided via a television or computer device in the SEE; adjust a sensitivity of a hearing aid or cochlear implant associated of the PUM; inject a therapeutically effective amount of insulin via an insulin pump of the PUM; activate a defibrillator or pace control device of a heart monitoring device of the PUM; dispense a therapeutically effective amount of a pharmaceutical agent from a storage vessel for consumption by the PUM; engage or disengage a lock on a door in the SEE; activate or silence an alarm disposed in the SEE or a caretaker area associated with the SEE; configure a granularity of reporting or a focus in the SEE of at least one environmental sensor; combinations thereof, and the like.

In various embodiments, various behaviors and deviations of behaviors from expected behaviors may be indicative of medical conditions that the system is permitted, via the HCP, to treat in addition or alternatively to suggesting a course of action to the PUM or other stakeholder or communication to the PUM or other stakeholder. For example, the system may be in communication with an insulin monitor and insulin pump, and may be permitted by the HCP to dispense insulin when the PUM exhibits aberrant behaviors associated with a blood sugar imbalance. For example, the system may be in communication with an implanted defibrillator or pacemaker, and may be permitted by the HCP to engage or reconfigure the imparted control signals to the heart of the PUM via the implanted cardiac device when the PUM exhibits aberrant behaviors associated with poor circulatory performance or changes exertion associated with different biological needs to blood flow. For example, the system may be in communication with an oxygen tank or supply system, and may be permitted by the HCP to adjust a flow rate into the room or a breathing apparatus when the PUM exhibits aberrant behaviors associated with low blood oxygen or changes exertion associated with different biological needs for oxygen.

In various embodiments, the hardware device may be engaged via messages or signals transmitted to a control device or computer associated with the hardware device. For example, when transmitting a message to a cellphone, a computing device may generate the message and format the message for transmission via a telephone network to the intended recipient. For example, a signal may be transmitted wirelessly or via wires to a motor associated with engaging a lock in a door when the PUM is identified as experiencing a dementia event and moving toward or establishing focus on a door or disengaging the lock when the PUM is observed as being lucid or uninterested in the door to selectively prevent or restrict the PUM from wandering from a particular area while improving (or not overly restricting) accessibility for others to the particular area while the PUM is located there.

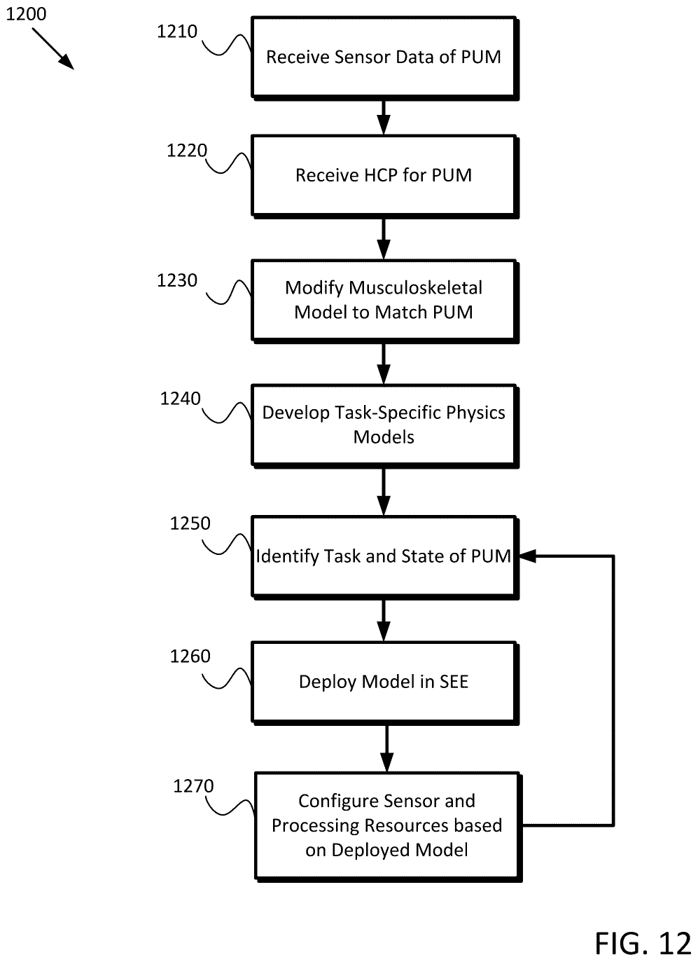

FIG. 12 is a flowchart of an example method 1200 for configuring one of both of the musculoskeletal models and sensor configurations used in monitoring and caring for a PUM in a SEE with respect to a PPE for improving the care of the PUM in a SEE, according to embodiments of the present disclosure. In some embodiments, the selection of the musculoskeletal model determines the sensor configuration used in the SEE. In some embodiments, the selection of a sensor configuration used in the SEE determines the musculoskeletal model used by the PPE. In some embodiments, the selection of the musculoskeletal model used by the PPE and the sensor configuration used in the SEE affect one another. Method 1200 begins at block 1210 where the system receives sensor data for a PUM (e.g., as described with respect to block 1110).

At block 1220, the system receives an HCP for the PUM. In various embodiments, the HCP may be received as one or more documents or electronic medical records (EMR) that are updated over time for the PUM. The HCP may include various directives, indicated contact persons, identification of health conditions, indications of physical attributes of the PUM, indications of mental acuity or psychological conditions of the PUM, indications of medications, indications of triggering and non-triggering actions, or the like that identify a plan of action for care of the PUM.

At block 1230, the system modifies a musculoskeletal model to match the PUM. In various embodiments, one or more baseline musculoskeletal models are used as a basis for representing the PUM, and are modified to match a gender, age, height, limb proportion, weight, fat/muscle ratio, injuries to joints or muscles, fusion or absence of any bones or limbs, presence of implanted devices, presence of external support devices (e.g., casts, braces, etc.) to provide a representation of the range of movement of the PUM.

At block 1240, the system develops one or more task-specific physics models for the musculoskeletal representation of the PUM. For example, a first task and a second task may each have separate physics models developed for the musculoskeletal representation of the PUM, which may include various elements in addition to the physical framework of the PUM. For example, a first model for walking may model various movements of the arms, legs, hips, and back of the PUM to represent the speed, stride length, step height, foot/ankle orientation, and other walking characteristics of the PUM. For example, a second model for carrying groceries may add masses to one or both hands of the PUM to represent the effect of the weight of the groceries on the movement of the PUM in the SEE while walking, affecting various walking characteristics. For example, a third model for putting away the groceries may include a weight distribution of the PUM relative with extending and retracting arms (e.g., reaching into a pantry or refrigerator) to identify instability or strain imposed by the task on the PUM.

As will be appreciated, different tasks involve different motions in different body parts of the PUM, and in some tasks, otherwise mobile body parts may be immobile. Accordingly, the various musculoskeletal models for a single PUM may be modeled with different numbers and locations of joints to thereby reduce the processing resources required to model the PUM performing a less complex task or set an appropriate amount of processing resources for a more involved task. For example, in a task-specific model for walking while not carrying any objects, the arms of the PUM may be modeled with shoulder and elbow joints to reflect the movement of the arms swinging while walking, but in task-specific model for walking not carrying a heavy objects in both arms (e.g., close to the chest), the arms of the PUM may be omitted and the center of gravity adjusted to account for the position of the arms and object being held. For example, in a task specific model for putting away groceries, in which the PUM stands in place, the leg joints of the PUM may be condensed into a single joint.

In some embodiments, the musculoskeletal model may be a null model (e.g., a model where joints are not modelled) for tasks or conditions that do not require joints to model (e.g., sleeping), or may be omitted in whole or in part for behaviors that are not correlated to (voluntary) movements or observations of the PUM. For example, a null model may be used to track the patterns or behaviors of the PUM in a sleeping task (e.g., falling asleep, waking, snoring, rolling over, etc.). For example, a null model may be used to track the comings and goings of a PUM in a SEE or a portion of the SEE without camera sensors (e.g., to determine when a PUM has been outside or away from home for long enough to be of concern).

At block 1250, the system identifies a task and a state of the PUM according to the sensor data (e.g., as described with respect to block 1120). In various embodiments, the task may be a series or pattern of several behaviors associated with an intent. For example, the task of getting dressed may include several behaviors associated with sitting down, standing up, taking off certain articles of clothing, putting on certain articles of clothing, etc. In some embodiments, the PPE adjusts a fidelity of the musculoskeletal representation for the PUM based on the HCP to adjust at least one parameter of the musculoskeletal representation to reflect one or more of: dimensions of body parts of the PUM; absence of at least one limb of the PUM; functional range of motion in at least one joint of the PUM; speed of motion for at least one joint-based motion for the PUM; force of motion of the at least one limb; and sequences of joint-based motions for the PUM that define motion frameworks employed by the PUM for movement and object manipulation in the SEE. In some embodiments, the PPE adjusts or bases the musculoskeletal representation at least in part on medical information included in the HCP for the PUM that indicates medical features including, but not limited to: a musculoskeletal ailment (e.g., broken bone; osteoporosis; fused bones; arthritis; a muscle, tendon or ligament tear, sprain or strain; etc.), a nervous system ailment (e.g., Parkinson's, Alzheimer's, Tourette's); a circulatory system ailment (e.g., arrhythmias; murmurs; pacemaker installation; etc.); a respiratory system ailment (e.g., bronchitis; pneumonia; cancer; full or partial removal of a lung; etc.); a gender of the PUM; an age of the PUM; and a physiological measurement of a body part of the PUM (e.g., a weight of an arm; a length of a thigh; a range of motion of a shoulder; etc.).

At block 1260, the system deploys the task-specific physical model in a digital twin of the SEE. For example, when the PUM is identified as intending to engage in a task in the SEE, the PPE may model the associated behaviors via the corresponding task-specific physical models. Accordingly, the PPE can switch from using a first musculoskeletal model to a second musculoskeletal model to model the series of predicted behaviors, and may do so in response to a various triggering events. These triggering events may include a change in granularity of the data received from a sensor (e.g., a sensor failure, a network data restriction, an increased contrast from the sensor, etc.), at least a portion of the PUM becoming unobservable or newly observable by the sensor, a different or new intent being identified for the PUM; and a behavior determined to satisfy the actionable threshold according to the HCP being identified (e.g., a triggering event).

In various embodiments, the PPE uses the selected model to generate motion frameworks within a series of predicted behaviors that define predefined sequences of actions based on a vector representation of joints in the musculoskeletal representation for the PUM with respect to one or more identified intended actions for the PUM to perform in the SEE. In various embodiments, a set of digital twins may evaluation more than one set of potential actions in parallel using the same or different models of the PUM to aid in predicting the actions that the PUM will engage in or potential outcomes of engaging those actions. For example, two digital twins may perform two alternative series of actions to achieve the same intent (e.g., taking a first path or a second path from a sofa to the kitchen) to identify if one of the series is more likely to result in harm to the PUM, and generate a response for a stakeholder to encourage the PUM avoid that series of actions (e.g., to avoid a tripping hazard present in the first path and absent from the second path).

At block 1270, the system configures sensor and processing resources in the actual SEE based on the deployed model. For example, when the model includes only large joints (e.g., elbows, shoulders, knees, hips, neck), the fidelity of any cameras in the SEE may be reduced to conserve power, processing resources, and transmission bandwidth compared to when the model includes small joints (e.g., wrists, ankles, fingers, sub-units of the neck or back, jaw) that may need greater fidelity to identify in the images collected from the SEE. For example, when the model includes joints in the upper body and not the lower body, cameras may be directed to focus on the upper body of the PUM in greater detail (and away from the lower body) compared to when using a model that includes the upper and lower bodies of the PUM.

Method 1200 may return to block 1250 to continue monitoring and simulating behaviors of the PUM using personalized task-specific models in the PPE.

FIG. 13 is a flowchart of an example method 1300 for maintaining and adjusting an HCP for the monitoring and care of a PUM in a SEE and adjusting a PPE accordingly for improving the care of a PUM in a SEE, according to embodiments of the present disclosure. Method 1300 begins at block 1310 where the system receives sensor data for a PUM (e.g., as described with respect to block 1110).

At block 1320, the system determines the state and behaviors of the PUM (e.g., as described with respect to block 1120).

At block 1330, the system models predicted behaviors of the PUM via a PPE and one or more personalized musculoskeletal models of the PUM. In some embodiments, the personalized musculoskeletal models are task-specific models (e.g., as generated and deployed per method 1200) that are modeled via digital twins within simulations of the SEE.

At block 1340, the system identifies trends in observed behaviors of the PUM from the sensor data received per block 1310. In various embodiments, the trends may include changes in behavior patterns over time or changes in adherence between predicted and performed behaviors over time. For example, a trend may be identified that identifies a reduced range of motion in a limb (e.g., reduced stride length, reduced step height) which may be correlated with a health condition changing over time (e.g., diminishing step size associated with reduced cardiovascular health). For example, a when a PUM is observed as more frequently or less frequently performing behaviors that match predicted behaviors of the PUM, a trend may be identified.

Various trend thresholds may be employed to ensure that a series of behaviors represents a trend rather than an aberration or a deviation within the bounds of what the system determines to be acceptable variation in behavior. For example, a PUM taking an atypical route from a sofa to the kitchen may be an aberration, but taking that same route at least N times of the next M times that the PUM walks from the sofa to the kitchen may be considered a trend.

In various embodiments, various trends in particularly identified behaviors of aspects of those behaviors may be observed for trends, which may be used to aid in the diagnosis and treatment of the underling conditions causing those behaviors or aspects of behavior. For example, the frequency or duration of dementia events or may be observed for trends, as can particular sub-types of dementia events (e.g., wandering, forgetfulness, talking to oneself). For example, a frequency, duration, or severity of jitter, seizures, Tourette's outbursts may be monitored for trends. These trends can be used to track the health of the PUM longitudinally, and may be tracked over time to identify a start of a behavior, an end of a behavior, or an inflection point in the progression of the behavior (e.g., an acceleration, deceleration in a frequency, duration, or severity trend). The different conditions, behaviors, and evaluation criteria for the various behaviors may be specified in the HCP for the PUM, and one of ordinary skill in the art will appreciate that these evaluation criteria may be specified according to known medical manuals and diagnostic techniques.

At block 1350, in response to identifying a trend in the PUM's behaviors, the system determines whether any changes in the HCP for the PUM occurred within a threshold time of the trend beginning, ending, or displaying an inflection. For example, when the PUM starts a new medication, stops taking a previously taken medication, or changes a dosage of a medication being taken, the behavior of the PUM may begin to change; however, as the effects of the medication change may not be immediately apparent, the system uses a time window of X days before the trend was identified to identify any changes in the HCP that may have contributed to the change in behavior.

In various embodiments, the time window may be configurable based on the type of change (e.g., increase or decrease), the behavior being changed, extent of the trend, user preferences (e.g., privacy policies), or the like and combinations thereof.

At block 1360, the system generates an update request for the HCP based on one or more of the identified change in the HCP or trend, a condition indicated in the HCP, and a type of behavior for which the trend was identified. For example, when the PUM is identified in the HCP as having dementia, and a worsening of symptoms is identified as a trend starting at day D, and the PUM began a course of blood thinning medication within X days before day D, the update request may identify this potential correlation to a medical practitioner or other stakeholder to potentially cease administration of the blood thinning medication, reduce a dosage of the blood thinning medication, switch the PUM to a different blood thinning medication, prescribe a secondary medication to counteract the potential negative effects of the blood thinning medication, or note the correlation for potential follow up.

For example, when treating or monitoring a condition that effects range of motion or mobility, the HCP update request can identify tasks that are no longer possible (or possible again) for a PUM to perform safely, and increase (or decrease) the amount of assistance allocated to the PUM, update a PPE or particular digital twin to reflect the changes in motor function of the PUM or extrapolate future motor function changes for predictive purposes, increase or decrease a sensitivity threshold for detecting potentially dangerous actions (e.g., fall risks), suggest additional exercises or stretching routines, and the like.

For example, when treating or monitoring a condition that includes tremor or jitter, various embodiments, the HCP update request identifies at least one treatment regimen selected for reducing jitter, such as, ceasing or reducing a dosage of a therapeutic agent prescribed to the PUM in the HCP associated with a side effect of inducing or intensifying jitter; suggesting re-diagnosis or re-analysis of a medical condition identified in the HCP that is associated with a symptom of jitter; suggesting diagnosis or analysis of a medical condition not identified in the HCP that is associated with the symptom of jitter; and ceasing or reducing a dietary component allowed for the PUM in the HCP that is associated with inducing or intensifying jitter.

The present disclosure contemplates that various other conditions and behaviors associated therewith may be monitored via the SEE to identify longitudinal trends in various behaviors to identify when symptoms begin, end, improve, or worsen. The particular treatments to cease, reduce, increase, or start (including re-start) may vary based on the condition being treated for, but may be specifically identified in the HCP for the PUM and identified therefrom based on the trend analysis. As these symptoms are traditionally monitored or evaluated via self-reporting, anecdotal observation, or infrequent evaluation, the presently described systems and methods offer improvements to the diagnosis, and potential treatment, of various maladies, conditions, and symptoms. Moreover, as humans are generally incapable of processing longitudinal data (e.g., not being able to identify a gradual worsening or improvement of symptoms), the present disclosure provides a solution that cannot be practically performed in the human mind.

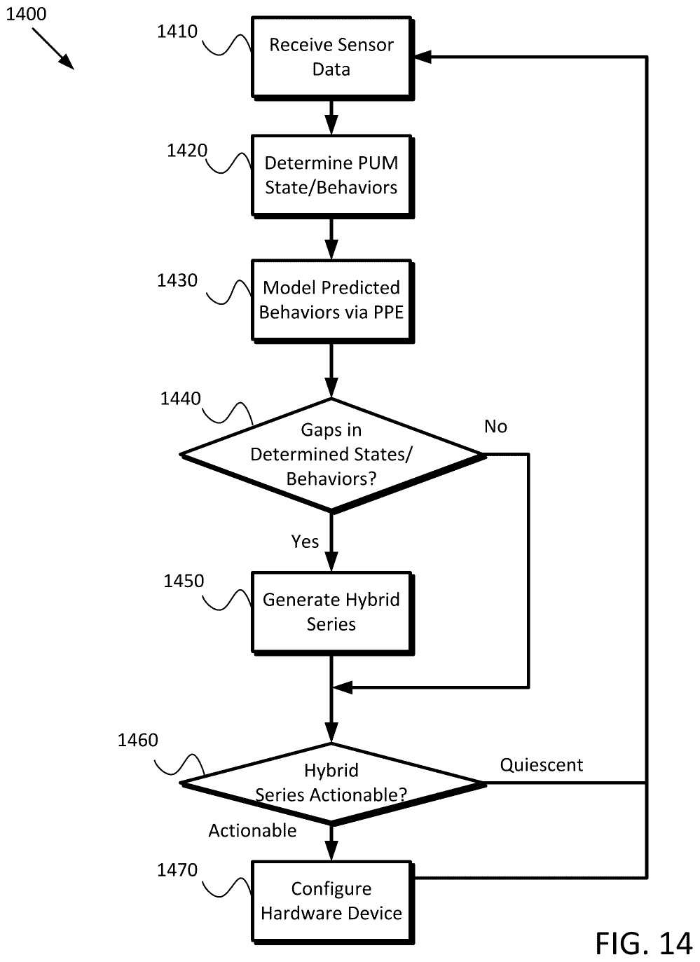

FIG. 14 is a flowchart of an example method 1400 for supplementing gaps in observed behaviors of a PUM in a SEE in data received from at least one sensor with data generated by a PPE for improving the care of a PUM in a SEE, according to embodiments of the present disclosure. Method 1400 begins at block 1410 where the system receives sensor data for a PUM (e.g., as described with respect to block 1110).

At block 1420, the system determines states and behaviors of the PUM (e.g., as described with respect to block 1120).

At block 1430, the system models precited behaviors of the PUM (e.g., as described with respect to block 1330).

At block 1440, the system determines whether any gaps exist in the determined states of behaviors determined per block 1420 relative to the modeled PUM per block 1430. When no gaps are identified, method 1400 proceeds to block 1460, and when a gap is identified, method 1400 proceeds to block 1450.

A gap in determined states/behaviors may be understood as a time period during which observed behaviors of the PUM are omitted from a series of observed behaviors described in the SEE. For example, gaps in the determined states or behaviors may occur when all or a portion of the PUM becomes unmonitorable if a sensor is obscured or malfunctions (e.g., creating a temporary or new dead zone), a network error or data corruption event occurs (e.g., dropping data or rendering data unusable), a sensor is misconfigured for monitoring the PUM (e.g., monitoring a second PUM instead of a first PUM, monitoring the wrong portion of the PUM, using a non-ideal granularity of reporting, etc.), a system lacks permissions to access certain data, a reporting frequency for data being less than a predefined threshold, and combinations thereof. In various embodiments, because the observed data are discretely reported over time, a practitioner may define one or time thresholds for a deficiency in observed data to qualify as a gap (e.g., at least 5 seconds, at least 10 seconds, at least 15 seconds, at least 30 seconds, at least 1 minute, at least 5 minutes, at least 10 minutes, etc.), and may set different time thresholds based on different times of day e.g., longer during sleeping hours versus waking hours), presence or number of persons in the SEE, HCP defined criteria, or the like. Additionally, the gap may include an upper bound threshold for which a time period is determined to be too long to define a gap for use in a hybrid analysis per method 1400 (e.g., no more than 10 seconds, no more than 15 seconds, no more than 30 seconds, no more than 1 minute, no more than 5 minutes, no more than 10 minutes, no more than 15 minutes, etc.).

Although the PPE and digital twins (and associated game theory games) may be able to model the actions of the PUM during a gap based on the intent, state or performed behaviors of the PUM before the gap occurred (e.g., predicted actions), and may supplement or revise the predicted actions using the intent, state or performed behaviors of the PUM the gap concludes, these modeled behaviors are generally treated separately from the observed behaviors. For example, two digital twins of the PUM may, using the same inputs, model different behaviors of the PUM for the same time period. Accordingly, the modeled data cannot be reliably substituted to fill the gaps in the observed data, which is exacerbated the larger the gap is.

The lack of observed data during gaps has been recognized as a computer-centric problems when dealing with remote sensor systems, the potential deficiencies in the simple substitution of modeled data will be appreciated. Additionally, the processing of such modeled data as though the data were observed data requires additional computing resources that may be wasted, accordingly, in some embodiments, the identification of a time period in which deficient sensor data are received to determine the state or behaviors of the PUM may also be conditioned on whether an actionable behavior was identified from the observed data.

For example, when the PUM travels through a sensor dead zone, the sensor data will be insufficient to determine a state or behavior of the PUM during the time that the PUM is within the sensor dead zone, and represents a gap in collected data from the SEE for monitoring the PUM. The time preceding the gap (e.g., from a first time to a second time) and the time following the gap (e.g., a third time to a fourth time) each provide sufficient data to determine the states and behaviors of the PUM, which may result in the identification of actionable behavior in either time period. If actionable behavior has been determined surrounding a time period otherwise sufficient to define a gap, the system may deem the gap in reporting insignificant or that the actionable behavior in the observed data override the identification of the gap, and method 1400 may proceed from block 1440 to block 1460.

At block 1450, the system generates a hybrid series of behaviors of the PUM to account for the gap in observed behaviors, using the modeled behaviors from one or more digital twins and the PPE. In various embodiments, the hybrid series is generated when the gap is of at least a predefined duration or amount of data, and represents a time period that is not preceded by or followed by an otherwise actionable behavior identified from the observed data.

In some embodiments, the hybrid series is generated by inserting a series of modeled behaviors of the PUM during the gap time between the series of performed behaviors observed before the gap and the series of performed behaviors observed after the gap. As will be appreciated, the PPE provides several models from several digital twins that may be run in parallel, and may generate multiple hybrid series using modeled behaviors from different parallel models. In some embodiments, the hybrid series is generated by overlaying a series of modeled behaviors of the PUM over available observed behaviors in the gap time between the series of performed behaviors observed before the gap and the series of performed behaviors observed after the gap. For example, if the gap includes some observed behaviors or states, but at a lower granularity than desired by the system for determining the intent, state or behavior of the PUM relative to the preceding or following times. As will be appreciated, the PPE provides several models from several digital twins that may be run in parallel, and may generate multiple hybrid series using modeled behaviors from different parallel models, and may select those modeled behaviors that match with any reported data within the gap (discarding or ignoring modeled behavior that do not match any received observational data).

At block 1460, the system determines whether a hybrid series of behaviors corresponds to an actionable behavior that was not identifiable from one or both of the observed or modeled behaviors individually. When an actionable behavior is identified from the hybrid series, method 1400 proceeds to block 1470, and otherwise returns to block 1410 for continued monitoring of the SEE and PUM when the hybrid series of behaviors corresponds to a quiescent behavior.

In various embodiments, thee hybrid series of observed and modeled behaviors remain separately observable from the constituent observed series of behaviors and modeled series of behaviors and are used to identify emergent behaviors that are actionable that would otherwise not be identified from just the constituent data.

For example, a behavior of the PUM moving about the SEE may be individually determined to be quiescent in a first time period (e.g., from t1 to t2), a second time period (e.g., from time t2 to t3), and a third time period (e.g., from time t3 to t4), but when viewed across those times (e.g., from t1 to t4) may demonstrate that the PUM is wandering back and forth in the SEE, which may be indicative of a dementia event, uncontrolled movement, or other actionable behavior.

At block 1470, the system (re)configures a hardware device for care of the PUM based on the actionable determination in block 1460 (e.g., as described with respect to block 1170). Method 1400 may return to block 1410 for continued monitoring of the SEE and PUM from block 1470.

Because the hybrid series that resulting in a determination of actionable behavior (per block 1460) includes observed behaviors or states that occurred after the gap, the contents of the response, or which stakeholders are identified to receive the response, may differ from predictive or reactive response or alerts from the system. For example, if a PUM is observed and modeled to have safely traversed an area of the SEE in which a tripping hazard is present, the response may indicate to a stakeholder to avoid that path in the future (e.g., as avoiding a hazard once may be due to good fortune), make another path more attractive in the future, make the observed/modeled path less attractive in the future, or combinations thereof.

### EXAMPLE COMPUTING DEVICE

All of the disclosed methods and procedures described in the present disclosure can be implemented using one or more computer programs or components. These components may be provided as a series of computer instructions on any conventional computer readable medium or machine readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

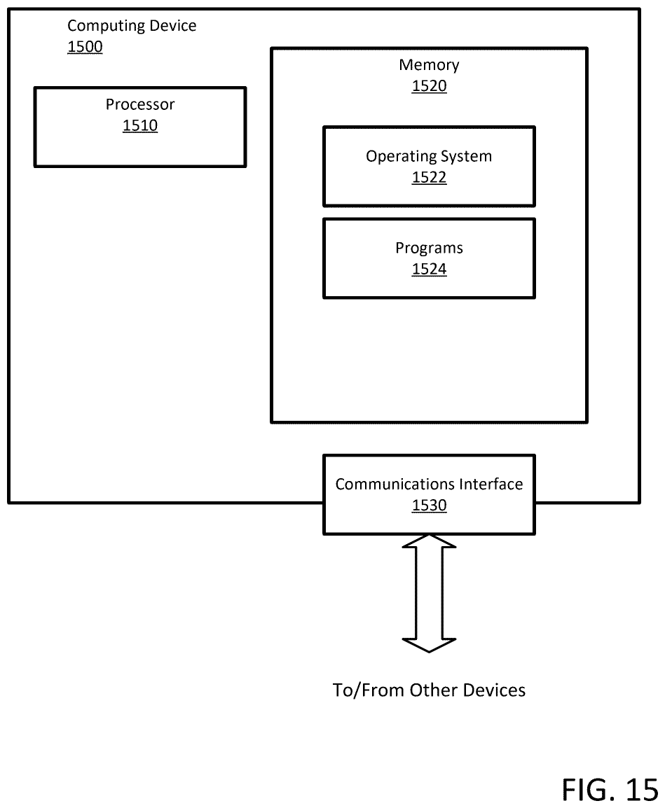

FIG. 15 illustrates an example computing device 1500, as may be used to provide one or more of the personalized physics engine, sensors, modules, hardware devices engaged for managing PUM or stakeholder behavior in response to actionable behaviors being identified or other systems described with respect to the sensor enabled environment or monitoring the person under monitoring, according to embodiments of the present disclosure. For example, the computing device 1500 may perform the operations set out in one or more of methods 1100, 1200, 1300, and 1400. The computing device 1500 may include at least one processor 1510, a memory 1520, and a communication interface 1530.

The processor 1510 may be any processing unit capable of performing the operations and procedures described in the present disclosure (e.g., methods 1100, 1200, 1300, and 1400). In various embodiments, the processor 1510 can represent a single processor, multiple processors, a processor with multiple cores, and combinations thereof.

The memory 1520 is an apparatus that may be either volatile or non-volatile memory and may include RAM, flash, cache, disk drives, and other computer readable memory storage devices. Although shown as a single entity, the memory 1520 may be divided into different memory storage elements such as RAM and one or more hard disk drives. As used herein, the memory 1520 is an example of a device that includes computer-readable storage media, and is not to be interpreted as transmission media or signals per se.

As shown, the memory 1520 includes various instructions that are executable by the processor 1510 to provide an operating system 1522 to manage various features of the computing device 1500 and one or more programs 1524 to provide various functionalities to users of the computing device 1500, which include one or more of the features and functionalities described in the present disclosure (e.g., methods 1100, 1200, 1300, and 1400). One of ordinary skill in the relevant art will recognize that different approaches can be taken in selecting or designing a program 1524 to perform the operations described herein, including choice of programming language, the operating system 1522 used by the computing device 1500, and the architecture of the processor 1510 and memory 1520. Accordingly, the person of ordinary skill in the relevant art will be able to select or design an appropriate program 1524 based on the details provided in the present disclosure.

Additionally, the memory 1520 may include one or more AM/ML models 1526 that interact with, are trained by, or are curated by the programs 1524. The AI/ML models 1526 may include generalized AI/ML models that are available for use (e.g., as a starting point) to various SEEs, as well as localized “edge” AI/ML models that are adjusted to reflect localized conditions in a particular SEE to better track and monitor a PUM, as described herein.

The communication interface 1530 facilitates communications between the computing device 1500 and other devices, including sensors in a SEE, which may also be computing devices as described in relation to FIG. 15. In various embodiments, the communication interface 1530 includes antennas for wireless communications and various wired communication ports. The computing device 1500 may also include or be in communication, via the communication interface 1530, one or more input devices (e.g., a keyboard, mouse, pen, touch input device, etc.) and one or more output devices (e.g., a display, speakers, a printer, etc.).

Although not explicitly shown in FIG. 15, it should be recognized that the computing device 1500 may be connected to one or more public or private networks via appropriate network connections via the communication interface 1530. It will also be recognized that software instructions may also be loaded into a non-transitory computer readable medium, such as the memory 1520, from an appropriate storage medium or via wired or wireless means.

Systems, methods, and apparatuses of the present disclosure may be implemented on a variety of devices, such as but not limited to IPUs, DPUs, CPUs, GPUs, ASICs, FPGAs, DSPs, or any other device capable of processing data. Instructions for performing the same may be provided as hardware or firmware on any computer-readable medium including volatile and non-volatile forms of memory. Particular implementations of techniques of the present disclosure may be structured in any number of ways, including but not limited to a modular program architecture, a monolithic program architecture, on a single device, and distributed across more than one device or processor.

Although certain figures and descriptions have been provided, many additional variations and modifications will be apparent to those of skill in the art. It will be appreciated that presenting all possible variations and modifications is an impractical task, and thus any sequence, particular structural or device implementation, or underlying technique of the present disclosure may be substituted or modified to meet the needs of particular implementations, and that doing so shall not depart from the scope of the present disclosure. It will therefore be appreciated that the examples presented herein are presented for illustrative purposes only, and are in no way intended to be limiting of a scope of the present disclosure. It will also be apparent to any individual of skill in the art that various embodiments described herein and elements thereof may be combined as needed to suit any particular implementation, and that doing so does not depart from the scope of the present disclosure. As such, the scope of the present disclosure is not to be understood as being limited by the figures or specification presented herein; the scope of the present disclosure should instead be understood in a context of the appended claims and their equivalents.

In addition to the embodiments described above, may examples of specific combinations are within the scope of the disclosure, some of which are detailed below with reference to the following numbered clauses.

Clause 1: A method, a system including a processor and a memory that stores instructions that are executable by the processor to perform operations of a method, or a memory device that stores instructions executable by a processor for performing operations of a method, the operations comprising: personalizing a musculoskeletal representation of a person for use in a personalized physics engine (PPE) for a person under monitoring (PUM) for care according to a Health Care Plan (HCP) to represent a movement capability of the PUM; receiving data from at least one sensor present in a sensor enabled environment (SEE) to identify behaviors of the PUM in the SEE at various times; identifying, via the data received from the at least one sensor, an intent for a first series of performed behaviors of the PUM in the SEE; modeling a series of predicted behaviors of the PUM in the SEE via the PPE based on the intent and a current behavior of the PUM in the SEE; identifying, via the data received from the at least one sensor, a second series of performed behaviors of the PUM in the SEE occurring after the first series of performed behaviors were performed; identifying a variation between the second series of performed behaviors and the series of predicted behaviors that satisfies an actionable threshold; in response to identifying that the variation satisfies the actionable threshold, updating a configuration of a hardware device associated with the SEE, wherein the hardware device is identified based on at least one of the intent and the variation.

Clause 2: The method, system, or device of any one of clauses 1-21, wherein updating the configuration the hardware device associated with the SEE includes an action selected from the group consisting of: adjusting a temperature of a thermostat for a heating ventilation and air conditioning unit associated with the SEE; adjusting a speed of a fan disposed in the SEE; turning off or on an appliance in the SEE; adjusting a volume, channel, brightness, contrast, or content item provided via a television or computer device in the SEE; adjusting a sensitivity of a hearing aid or cochlear implant associated of the PUM; injecting a therapeutically effective amount of insulin via an insulin pump of the PUM; activating a defibrillator or pace control device of a heart monitoring device of the PUM; adjusting a brightness of a light in the SEE; engaging or disengaging a lock on a door in the SEE; activating or silencing an alarm disposed in the SEE or a caretaker area associated with the SEE; causing a speaker disposed in the SEE or the caretaker area associated with the SEE to convey an audio message; configuring a granularity of reporting or a focus in the SEE of the at least one sensor; and transmitting a message to a telephone, pager or computer device associated with a stakeholder for care of the PUM.

Clause 3: The method, system, or device of any one of clauses 1-21, wherein the stakeholder is selected based on the intent and based on an association of the stakeholder with achieving the intent on behalf of the PUM, the stakeholder being selected from the group consisting of: the PUM; a family member of the PUM; a friend of the PUM; a neighbor of the PUM; a designated health, wellness, or safety contact of the PUM; a doctor; a nurse; a healthcare assistant; an emergency services provider; a care animal; and a living facility attendant; or any other person or entity acting as a carer to the PUM.

Clause 4: The method, system, or device of any one of clauses 1-21, wherein engaging the hardware device includes adjusting a granularity of the at least one sensor to continue producing future data with an output characteristic selected from the group consisting of: a different focus or position of the at least one sensor in the SEE; a different rate of data transmission from the at least one sensor; a different sensing capability; and a different fidelity of data collection by the at least one sensor.

Cause 5: The method, system, or device of any one of clauses 1-21, wherein personalization of the musculoskeletal representation is based at least in part on observed behaviors identified via a machine learning or artificial intelligence (AI/ML) model observing historically collected data by the at least one sensor for the PUM to identify the movement capability of the PUM.

Clause 6: The method, system, or device of any one of clauses 1-21, wherein personalization of the musculoskeletal representation is based at least in part on medical information included in the HCP for the PUM indicating a medical feature selected from the group consisting of: a musculoskeletal ailment; a nervous system ailment; a circulatory system ailment; a respiratory system ailment; a gender of the PUM; an age of the PUM; and a physiological measurement of a body part of the PUM.

Clause 7: The method, system, or device of any one of clauses 1-21, wherein the PPE uses at least one game theory game to determine which one behavior of a plurality of potential movements follows a particular movement in the series of predicted movements that can form at least one behavior based on a reward framework for matching behaviors to the intent.

Clause 8: The method, system, or device of any one of clauses 1-21, wherein the PPE generates motion frameworks within the series of predicted behaviors that define predefined sequences of actions based on a vector representation of joints in the musculoskeletal representation for the PUM with respect to one or more identified intended actions for the PUM to perform in the SEE.

Clause 9: The method, system, or device of any one of clauses 1-21, wherein personalizing the musculoskeletal representation for the PUM adjusts a fidelity of the musculoskeletal representation for the PUM based on the HCP to adjust at least one parameter of the musculoskeletal representation to reflect the at least one parameter as applied to the PUM via modeled laws of physics, the at least one parameter selected from the group consisting of: dimensions of body parts of the PUM; absence of at least one limb of the PUM; functional range of motion in at least one joint of the PUM; speed of motion for at least one joint-based motion for the PUM; force of motion of the at least one limb; and sequences of joint-based motions for the PUM that define motion frameworks employed by the PUM for movement and object manipulation in the SEE.

Clause 10: The method, system, or device of any one of clauses 1-21, wherein the first series of performed behaviors identified from the data received from the at least one sensor represent behaviors of the PUM from a first time to a second time, and from a third time to a fourth time, omitting description of behaviors of the PUM from the second time to the third time, wherein the series of performed behaviors are determined to correspond to quiescent behaviors for the PUM, the operations further comprising: inserting the series of predicted behaviors of the PUM modeled from the second time to the third time into the first series of performed behaviors to produce a series of hybrid behaviors describing behaviors of the PUM inclusively from the first time through the fourth time; and in response to determining that the series of hybrid behaviors correspond to an actionable behavior, updating a configuration of a second hardware device associated with the SEE, wherein the second hardware device is identified based on at least one of the intent and the variation.

Clause 11: The method, system, or device of any one of clauses 1-21, wherein the PPE models the series of predicted behaviors from the second time to the third time based on the series of performed behaviors identified from the data received from the at least one sensor from the first time to the second time, and from the third time to the fourth time, wherein a time period between the second time and the third time exceeds a predefined threshold.

Clause 12: The method, system, or device of any one of clauses 1-21, wherein the PPE models the musculoskeletal representation of the PUM represented in one or more digital twins of the PUM.

Clause 13: The method, system, or device of any one of clauses 1-21, wherein the intent of the first series of behaviors is determined based on a focal point in the SEE and at least one of a gaze of the PUM relative to the focal point and a direction of motion of the PUM relative to the focal point made during the first series of performed behaviors.

Clause 14: The method, system, or device of any one of clauses 1-21, the operations further comprising: evaluating the first series of performed behaviors, the second series of performed behaviors, and the series of predicted behaviors for an HCP trigger identified in the HCP that satisfies an alerting threshold; and generating an alert in response to detecting the HCP trigger.

Clause 15: The method, system, or device of any one of clauses 1-21, wherein the HCP trigger corresponds to movement of a joint of the PUM identified in the HCP with pain or reduced efficacy of movement for the PUM, wherein the alert includes a suggested alternative behavior for achieving the intent with reduced pain or with improved efficacy of movement for the PUM.

Clause 16: The method, system, or device of any one of clauses 1-21, wherein modeling the series of predicted behaviors of the PUM in the SEE informed by the PPE identifies a path through the SEE, the method further comprising: evaluating the path for presence of an HCP trigger identified in the HCP; and generating alerts in response to detecting the HCP trigger.

Clause 17: The method, system, or device of any one of clauses 1-21, wherein the HCP trigger is selected from the group consisting of: a tripping or fall hazard; a repeated movement through the SEE towards and away from a focal point identified with the intent; a destination point of the series of predicted behaviors not associated with the focal point identified with the intent; a false-start motion in the second series of performed behaviors absent from the series of predicted behaviors; and a pain-inducing motion in the second series of performed behaviors absent from the series of predicted behaviors.

Clause 18: The method, system, or device of any one of clauses 1-21, wherein monitoring systems that include the PPE identify the PUM as experiencing jitter, wherein the PPE is configured to ignore movement below a gross motor threshold when identifying whether the variation between the second series of performed behaviors and the series of predicted behaviors exceeds the actionable threshold.

Clause 19: The method, system, or device of any one of clauses 1-21, wherein monitoring systems identify the PUM as experiencing jitter, wherein the PPE is configured to monitor a severity or frequency of jitter as part of monitoring care of the PUM, the operations further comprising: generating an HCP update request in response to the severity or frequency of jitter changing more than a threshold amount from a first time to a second time.

Clause 20: The method, system, or device of any one of clauses 1-21, wherein the HCP update request identifies at least one treatment regimen selected for reducing jitter from the group consisting of: ceasing or reducing a dosage of a therapeutic agent prescribed to the PUM in the HCP associated with a side effect of inducing or intensifying jitter; suggesting re-diagnosis or re-analysis of a medical condition identified in the HCP that is associated with a symptom of jitter; suggesting diagnosis or analysis of a medical condition not identified in the HCP that is associated with the symptom of jitter; and ceasing or reducing a dietary component allowed for the PUM in the HCP that is associated with inducing or intensifying jitter.

Clause 21: The method, system, or device of any one of clauses 1-21, wherein the musculoskeletal representation used by the PPE includes at least a first musculoskeletal model and a second musculoskeletal model of the PUM, wherein the first musculoskeletal model includes a different number of joints modeled for the PUM than the second musculoskeletal model includes, wherein the PPE selects to use the first musculoskeletal model rather than the second musculoskeletal model in modeling the series of predicted behaviors based on the intent, the current behavior of the PUM, and a current sensor configuration in the SEE, the operations further comprising: switching, in the PPE from use of the first musculoskeletal model to the second musculoskeletal model to model the series of predicted behaviors in response to a triggering event selected from the group consisting of: a change in granularity of the data received from the at least one sensor; at least a portion of the PUM becoming unobservable or newly observable by the at least one sensor; a different intent being identified from the second series of performed behaviors than the intent identified from the first series of performed behaviors; and a behavior determined to satisfy the actionable threshold according to the HCP being identified from the second series of performed behaviors or the series of predicted behaviors.

Certain terms are used throughout the description and claims to refer to particular features or components. As one skilled in the art will appreciate, different persons may refer to the same feature or component by different names. This document does not intend to distinguish between components or features that differ in name but not function.

As used herein, the term “optimize” and variations thereof, is used in a sense understood by data scientists to refer to actions taken for continual improvement of a system relative to a goal. An optimized value will be understood to represent “near-best” value for a particular reward framework, which may oscillate around a local maximum or a global maximum for a “best” value or set of values, which may change as the goal changes or as input conditions change. Accordingly, an optimal solution for a first goal at a particular time may be suboptimal for a second goal at that time or suboptimal for the first goal at a later time.

As used herein, “about,” “approximately” and “substantially” are understood to refer to numbers in a range of the referenced number, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, the term “or” is to be interpreted in the inclusive sense and not the exclusive sense unless explicitly stated otherwise or when clear from the context. Accordingly, recitation of “A or B” is intended to cover the sets of A, B, and A-B, where the sets may include one or multiple instances of a particular member (e.g., A-A, A-A-A, A-A-B, etc.) and any ordering thereof.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, A-C, and A-B-C, where the sets may include one or multiple instances of a particular member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

As used in the present disclosure, the term “determining” encompasses a variety of actions that may include calculating, computing, processing, deriving, investigating, identifying, looking up (e.g., via a table, database, or other data structure), ascertaining, receiving (e.g., receiving information), accessing (e.g., accessing data in a memory), retrieving, resolving, selecting, choosing, establishing, and the like.

Without further elaboration, it is believed that one skilled in the art can use the preceding description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112(f) unless the element is expressly recited using the phrase “means for” or “step for”. All structural and functional equivalents to the elements of the various embodiments described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

It should be understood that various changes and modifications to the examples described here will be apparent to those skilled in the art. Such changes and modifications can be made without departing from the spirit and scope of the present subject matter and without diminishing its intended advantages. It is therefore intended that such changes and modifications be covered by the appended claims.

What is claimed is:

1. A method, comprising:
personalizing a musculoskeletal representation of a person for use in a personalized physics engine (PPE) for a person under monitoring (PUM) for care according to a Health Care Plan (HCP) to represent a movement capability of the PUM;
receiving data from at least one sensor present in a sensor enabled environment (SEE) to identify behaviors of the PUM in the SEE at various times;
identifying, via the data received from the at least one sensor, an intent for a first series of performed behaviors of the PUM in the SEE;
modeling a series of predicted behaviors of the PUM in the SEE via the PPE based on the intent and a current behavior of the PUM in the SEE;
identifying, via the data received from the at least one sensor, a second series of performed behaviors of the PUM in the SEE occurring after the first series of performed behaviors were performed;
identifying a variation between the second series of performed behaviors and the series of predicted behaviors that satisfies an actionable threshold; and
in response to identifying that the variation satisfies the actionable threshold, updating a configuration of a hardware device associated with the SEE, wherein the hardware device is identified based on at least one of the intent and the variation.

2. The method of claim 1, wherein updating the configuration the hardware device associated with the SEE includes an action selected from the group consisting of:
adjusting a temperature of a thermostat for a heating ventilation and air conditioning unit associated with the SEE;
adjusting a speed of a fan disposed in the SEE;
turning off or on an appliance in the SEE;
adjusting a volume, channel, brightness, contrast, or content item provided via a television or computer device in the SEE;
adjusting a brightness of a light in the SEE;
engaging or disengaging a lock on a door in the SEE;
activating or silencing an alarm disposed in the SEE or a caretaker area associated with the SEE;
causing a speaker disposed in the SEE or the caretaker area associated with the SEE to convey an audio message;
configuring a granularity of reporting or a focus in the SEE of the at least one sensor; and
transmitting a message to a telephone, pager or computer device associated with a stakeholder for care of the PUM.

3. The method of claim 2, wherein the stakeholder is selected based on the intent and based on a relationship of the stakeholder with achieving the intent on behalf of the PUM, the stakeholder being selected from the group consisting of:
the PUM;
a family member of the PUM;
a neighbor of the PUM;
a friend of the PUM;
a designated health, wellness, or safety contact of the PUM;
a doctor;
a nurse;
a healthcare assistant;
an emergency services provider;
a care animal; and
a living facility attendant.

4. The method of claim 1, wherein engaging the hardware device includes adjusting a granularity of the at least one sensor to continue producing future data with an output characteristic selected from the group consisting of:
a different focus or position of the at least one sensor in the SEE;
a different rate of data transmission from the at least one sensor,
a different sensing capability; and
a different fidelity of data collection by the at least one sensor.

5. The method of claim 1, wherein personalization of the musculoskeletal representation is based at least in part on observed behaviors identified via a machine learning or artificial intelligence (AI/ML) model observing historically collected data by the at least one sensor for the PUM to identify the movement capability of the PUM.

6. The method of claim 1, wherein personalization of the musculoskeletal representation is based at least in part on medical information included in the HCP for the PUM indicating a medical feature selected from the group consisting of:
a musculoskeletal ailment;
a nervous system ailment;
a circulatory system ailment;
a respiratory system ailment;
a gender of the PUM;
an age of the PUM; and
a physiological measurement of a body part of the PUM.

7. The method of claim 1, wherein the PPE uses at least one game theory game to determine which one behavior of a plurality of potential movements follows a particular movement in the series of predicted movements that can form at least one behavior based on a reward framework for matching behaviors to the intent.

8. The method of claim 1, wherein the PPE generates motion frameworks within the series of predicted behaviors that define predefined sequences of actions based on a vector representation of joints in the musculoskeletal representation for the PUM with respect to one or more identified intended actions for the PUM to perform in the SEE.

9. The method of claim 1, wherein personalizing the musculoskeletal representation for the PUM adjusts a fidelity of the musculoskeletal representation for the PUM based on the HCP to adjust at least one parameter of the musculoskeletal representation to reflect the at least one parameter as applied to the PUM via modeled laws of physics, the at least one parameter selected from the group consisting of:
dimensions of body parts of the PUM;
absence of at least one limb of the PUM;
functional range of motion in at least one joint of the PUM;
speed of motion for at least one joint-based motion for the PUM;
force of motion of the at least one limb; and
sequences of joint-based motions for the PUM that define motion frameworks employed by the PUM for movement and object manipulation in the SEE.

10. The method of claim 1, wherein the first series of performed behaviors identified from the data received from the at least one sensor represent behaviors of the PUM from a first time to a second time, and from a third time to a fourth time, omitting description of behaviors of the PUM from the second time to the third time, wherein the series of performed behaviors are determined to correspond to quiescent behaviors for the PUM, the method further comprising:
inserting the series of predicted behaviors of the PUM modeled from the second time to the third time into the first series of performed behaviors to produce a series of hybrid behaviors describing behaviors of the PUM inclusively from the first time through the fourth time; and
in response to determining that the series of hybrid behaviors correspond to an actionable behavior, updating a configuration of a second hardware device associated with the SEE, wherein the second hardware device is identified based on at least one of the intent and the variation.

11. The method of claim 10, wherein the PPE models the series of predicted behaviors from the second time to the third time based on the series of performed behaviors identified from the data received from the at least one sensor from the first time to the second time, and from the third time to the fourth time, wherein a time period between the second time and the third time exceeds a predefined threshold.

12. The method of claim 1, wherein the PPE models the musculoskeletal representation of the PUM represented in one or more digital twins of the PUM.

13. The method of claim 1, wherein the intent of the first series of behaviors is determined based on a focal point in the SEE and at least one of a gaze of the PUM relative to the focal point and a direction of motion of the PUM relative to the focal point made during the first series of performed behaviors.

14. The method of claim 1, further comprising:
evaluating the first series of performed behaviors, the second series of performed behaviors, and the series of predicted behaviors for an HCP trigger identified in the HCP that satisfies an alerting threshold; and
generating an alert in response to detecting the HCP trigger.

15. The method of claim 14, wherein the HCP trigger corresponds to movement of a joint of the PUM identified in the HCP with pain or reduced efficacy of movement for the PUM, wherein the alert includes a suggested alternative behavior for achieving the intent with reduced pain or with improved efficacy of movement for the PUM.

16. The method of claim 1, wherein modeling the series of predicted behaviors of the PUM in the SEE informed by the PPE identifies a path through the SEE, the method further comprising:
evaluating the path for presence of an HCP trigger identified in the HCP; and
generating alerts in response to detecting the HCP trigger.

17. The method of claim 16, wherein the HCP trigger is selected from the group consisting of:
a tripping or fall hazard;
a repeated movement through the SEE towards and away from a focal point identified with the intent;
a destination point of the series of predicted behaviors not associated with the focal point identified with the intent;
a false-start motion in the second series of performed behaviors absent from the series of predicted behaviors; and
a pain-inducing motion in the second series of performed behaviors absent from the series of predicted behaviors.

18. The method of claim 1, wherein monitoring systems that include the PPE identify the PUM as experiencing jitter, wherein the PPE is configured to ignore movement below a gross motor threshold when identifying whether the variation between the second series of performed behaviors and the series of predicted behaviors exceeds the actionable threshold.

19. The method of claim 1, wherein monitoring systems identify the PUM as experiencing jitter, wherein the PPE is configured to monitor a severity or frequency of jitter as part of monitoring care of the PUM, the method further comprising:
generating an HCP update request in response to the severity or frequency of jitter changing more than a threshold amount from a first time to a second time.

20. The method of claim 19, wherein the HCP update request identifies at least one treatment regimen selected for reducing jitter from the group consisting of:
ceasing or reducing a dosage of a therapeutic agent prescribed to the PUM in the HCP associated with a side effect of inducing or intensifying jitter;
suggesting re-diagnosis or re-analysis of a medical condition identified in the HCP that is associated with a symptom of jitter;
suggesting diagnosis or analysis of a medical condition not identified in the HCP that is associated with the symptom of jitter; and
ceasing or reducing a dietary component allowed for the PUM in the HCP that is associated with inducing or intensifying jitter.

21. The method of claim 1, wherein the musculoskeletal representation used by the PPE includes at least a first musculoskeletal model and a second musculoskeletal model of the PUM, wherein the first musculoskeletal model includes a different number of joints modeled for the PUM than the second musculoskeletal model includes, wherein the PPE selects to use the first musculoskeletal model rather than the second musculoskeletal model in modeling the series of predicted behaviors based on the intent, the current behavior of the PUM, and a current sensor configuration in the SEE, the method further comprising:
switching, in the PPE from use of the first musculoskeletal model to the second musculoskeletal model to model the series of predicted behaviors in response to a triggering event selected from the group consisting of:
a change in granularity of the data received from the at least one sensor;
at least a portion of the PUM becoming unobservable or newly observable by the at least one sensor;
a different intent being identified from the second series of performed behaviors than the intent identified from the first series of performed behaviors; and
a behavior determined to satisfy the actionable threshold according to the HCP being identified from the second series of performed behaviors or the series of predicted behaviors.

22. A system, comprising:
a processor; and
a memory, including instructions that, when executed by the processor, perform operations that include:
personalizing a musculoskeletal representation of a person for use in a personalized physics engine (PPE) for a person under monitoring (PUM) for care according to a Health Care Plan (HCP) to represent a movement capability of the PUM;
receiving data from at least one sensor present in a sensor enabled environment (SEE) to identify behaviors of the PUM in the SEE at various times;
identifying, via the data received from the at least one sensor, an intent for a first series of performed behaviors of the PUM in the SEE;
modeling a series of predicted behaviors of the PUM in the SEE via the PPE based on the intent and a current behavior of the PUM in the SEE;
identifying, via the data received from the at least one sensor, a second series of performed behaviors of the PUM in the SEE occurring after the first series of performed behaviors were performed;
identifying a variation between the second series of performed behaviors and the series of predicted behaviors that satisfies an actionable threshold; and
in response to identifying that the variation satisfies the actionable threshold, updating a configuration of a hardware device associated with the SEE, wherein the hardware device is identified based on at least one of the intent and the variation.
